SEQUENCE LISTING (14 sequences)

SEQ ID NO: 1 - DNA, 960 bp, Lactobacillus delbrueckii
atgaaacggattggtattttgactagcggcggtgacgcccctggtatgaatgcagccgtaagagccgtaaccagagtcgcgatcgcgaacggtttggaagttttcggtatcagatacggttttgcaggtttggttgcgggcgacattttcccattggaaagtgaagacgtagcccacttgatcaatgtttccggtaccttcctctactctgcgcgttatcctgaatttgcagaagaagaaggacagctggctggtattgaacaattgaagaagcacggcatcgatgctgttgtcgttattggtggggatggttcctaccatggcgccctgcagctgacccgccacggcttcaactcaattggcctgccaggcacgatcgacaacgatatcccttacacggatgccacgatcggctatgacacggcctgcatgacggcaatggacgcgatcgacaagatccgtgacactgcttctagccaccaccgcgtcttcattgtcaacgtaatgggccgcaactgtggtgacatcgctatgcgcgtcggcgtagcctgcggggcggacgcgatcgtcattcctgaaagaccatatgatgtcgaagaaatcgccaaccggctcaagcaagcccaggaaagcggcaaggaccacggtttggtagttgttgctgaaggcgtaatgaccgctgaccaattcatggctgaactgaagaagtatggtgacttcgacgtccgggccaatgttttgggtcacatgcagcggggcggcacgccaactgtttctgaccgggtgctggcttccaagctgggcagcgaagccgttcaccttcttttggaaggcaagggcggcctggctgtcggcattgaaaacggcaaggttacttcacacgacatccttgatttatttgatgagtctcacaggggtgactatgacttgttaaagctcaacgcagatttatcaagatag

SEQ ID NO: 2 - protein, 319 aa, Lactobacillus delbrueckii
MKRIGILTSGGDAPGMNAAVRAVTRVAIANGLEVFGIRYGFAGLVAGDIFPLESEDVAHLINVSGTFLYSARYPEFAEEEGQLAGIEQLKKHGIDAVVVIGGDGSYHGALQLTRHGFNSIGLPGTIDNDIPYTDATIGYDTACMTAMDAIDKIRDTASSHHRVFIVNVMGRNCGDIAMRVGVACGADAIVIPERPYDVEEIANRLKQAQESGKDHGLVVVAEGVMTADQFMAELKKYGDFDVRANVLGHMQRGGTPTVSDRVLASKLGSEAVHLLLEGKGGLAVGIENGKVTSHDILDLFDESHRGDYDLLKLNADLSR

SEQ ID NO: 3 - DNA, 1770 bp, Lactobacillus delbrueckii
atgaaaaaaacaaagattgttagtactttagggccagcttcagacgatattgaaactattaccaagttagccgaagcaggcgcaaacgtattccgtttcaacttctcacacggtaaccacgaagaacacttggcaagaatgaacatggttcgtgaagttgaaaagaagactggcaagcttttgggcatcgctttggacaccaagggtgctgaaatcagaaccactgaccaagaaggcggcaagttcactatcaacactggtgacgaaatccgcgtgtcaatggacgcaaccaaggccggcaacaaggacatgatccacgttacctacccaggtctgttcgacgacactcacgtaggcggcactgtattgatcgacgacggtgctgttggtttgactatcaaggccaaggacgaagaaaagcgcgaattggtttgtgaagctcaaaacactggtgtcatcggctcaaagaagggtgttaacgctccaggtgttgaaatccgcctcccagggattactgaaaaggacactgacgacatccgctttggtttgaagcacggtattaacttcatctttgcttcatttgtacgtaaggctcaagacgttcttgacattcgcgcactttgcgaagaagctaacgcatcatacgttaagatcttcccaaagattgaatcacaagaaggtattgacaacatcgacgaaatcttgcaagtttcagatggtttgatggttgcccgtggtgacatgggtgttgaaatcccattcatcaacgtgccatttgttcaaaagactttgatcaagaagtgcaacgctttgggcaagccagttatcactgctactcaaatgctggactcaatgcaagaaaacccacgtccaacccgtgccgaagtaactgacgttgctaacgccgttcttgacggtactgacgcaactatgctgtcaggtgaatcagcaaacggtttgtacccagtacaatcagttcaagctatgcacgacatcgatgttcggactgaaaaggaattggacacccggaacactctggctctgcaacgctttgaagaatacaagggctcaaacgttactgaagctatcggtgaatcagttgtccgcactgctcaagaactgggcgttaagactatcatcgctgctactagctccggctacacagctcgtatgatctccaagtaccgtccagacgcaaccatcgttgccttgactttcgacgaaaagatccaacactcattgggtatcgtttggggcgttgaaccagttttggcaaagaaaccttcaaacactgacgaaatgttcgaagaagctgcccgcgtagctaaggaacacggtttcgttaaggatggcgacctggtaatcatcgttgccggcgtaccattcggccaatcaggtactactaacttgatgaagctgcaaatcatcggcaaccaacttgctcaaggtttgggcgtaggcactggctcagttatcggcaaggctgttgttgcgaacagcgctgaagaagccaacgctaaggttcacgaaggcgacatcctggtagctaagactactgacaaggactacatgccagctatcaagaaggccagcggtatgatcgttgaagcttccggcttgaccagccacgcagctgttgtcggcgtatcactcggcattccagttgttgtcggtgttgctgacgcaacttcaaagatcgctgacggctcaactttgactgttgacgcacgtcgcggcgcaatttaccaaggtgaagtttcaaacctgtaa

SEQ ID NO: 4 - protein, 589 aa, Lactobacillus delbrueckii
MKKTKIVSTLGPASDDIETITKLAEAGANVFRFNFSHGNHEEHLARMNMVREVEKKTGKLLGIALDTKGAEIRTTDQEGGKFTINTGDEIRVSMDATKAGNKDMIHVTYPGLFDDTHVGGTVLIDDGAVGLTIKAKDEEKRELVCEAQNTGVIGSKKGVNAPGVEIRLPGITEKDTDDIRFGLKHGINFIFASFVRKAQDVLDIRALCEEANASYVKIFPKIESQEGIDNIDEILQVSDGLMVARGDMGVEIPFINVPFVQKTLIKKCNALGKPVITATQMLDSMQENPRPTRAEVTDVANAVLDGTDATMLSGESANGLYPVQSVQAMHDIDVRTEKELDTRNTLALQRFEEYKGSNVTEAIGESVVRTAQELGVKTIIAATSSGYTARMISKYRPDATIVALTFDEKIQHSLGIVWGVEPVLAKKPSNTDEMFEEAARVAKEHGFVKDGDLVIIVAGVPFGQSGTTNLMKLQIIGNQLAQGLGVGTGSVIGKAVVANSAEEANAKVHEGDILVAKTTDKDYMPAIKKASGMIVEASGLTSHAAVVGVSLGIPVVVGVADATSKIADGSTLTVDARRGAIYQGEVSNL

SEQ ID NO: 5 - DNA, 27 bp, Artificial Sequence
  Description of Artificial Sequence  Synthetic Primer
aggcgcgccaccatgaaacggattggt

SEQ ID NO: 6 - DNA, 27 bp, Artificial Sequence
  Description of Artificial Sequence  Synthetic Primer
cgcctgcaggctatcttgataaatctg

SEQ ID NO: 7 - DNA, 27 bp, Artificial Sequence
  Description of Artificial Sequence  Synthetic Primer
aggcgcgccaccatgaaaaaaacaaag

SEQ ID NO: 8 - DNA, 25 bp, Artificial Sequence
  Description of Artificial Sequence  Synthetic Primer
cgcctgcaggttacaggtttgaaac

SEQ ID NO: 9 - DNA, 2829 bp, Schizosaccharomyces pombe
atgagtggagaaaccgtgcatggaatttcgtgctactctgttgttgcaaacactgaggacacatataatcagactctcgacttctaccaaaagcttggcttcaagaaggttgccagcttcggtaccagcgattctgacaatgcccgtgtttgcaacgagtctctccgtgaggactggatgcatgtcgctggaaacaactctgctgaaagcgtcaccatcaagttccgtttagtccccggtgaattgagcctttcccccgctgccgaagattctgaatggcgcggacaaaagagttctcttgttttttactatcccaacttgcttgacttgcttaagcaactcagtgccgatgccattaaataccaagctttccccaacgagaagaagcctgatgaggtttatgtcgaggatcccttgggtaatttgattggcttctccgaccgttacaatcctttcgcccatgctaaccttaagaagagcgaggagtctggtgctgcttctaacctcgagagcggtttggctactcccgtcgttgagactctcaagaaagctaccacttctgacaagcctgcaggtgtcaagaagaagattgcagttatgaccagtggtggtgactcccctggtatgaacgctgttgttcgtgccgtcgctcgtattgccattcaccgtggttgtgatgctttcgctatttatgaaggttatgaaggtcttgttcaaggtggtgatatgatcaagcaattgcaatggggtgatgtccgtggttggcttgctgagggtggtactcttattggtaccgctcgttgtatggctttccgtgagcgtcctggtcgtcttcgtgctgccaagaaccttatttccgctggtattgattccattattgtttgcggtggtgatggttctttgaccggtgctgatatcttccgttctgactggcccggtttggttaaagagttggaggacaccaaggctattactcccgagcaagccaagctctaccgccatcttaccattgtcggtttggtcggttcaatcgataacgatatgtcttcaactgacgttactatcggtgccttctcttctcttcaccgtatttgcgaagccgtcgactccatttcttctactgctatttcccattctcgtgctttcatcgttgaagtcatgggtcgtcattgcggttggttggctgttttagctgcattggctaccggtgctgatttcgtctttatccctgagagacctgctgaagtcggcaaatggcaagacgaattgtgcaattctttgagctctgtccgtaagttgggcaagagaaagtcaattgttattgttgcagagggtgctattgactccgagcttaaccacatttctcccgaagacatcaagaacttgttagttgagcgccttcacttggacactcgtgtcaccactctcggtcacgttcaacgtggtggtattccttgtgcttatgaccgtatgcttgctacccttcaaggtgttgatgccgttgatgctgttcttgcctctacccctgatactccatctcccatgattgctatcaatggtaacaagattaaccgcaagcctttgatggaggctgttaagttgactcatgaggttgctgatgctattgagaagaagcagttcgctcatgctatggagctccgtgaccccgaatttgctgattacttacacacttgggaaggtactacctttattgaagacgagtcacacttcgttcccaaggacgagagaatgcgtgtcgccatcatccatgttggtgctcccgctggtggtatgaactctgctactcgtgctgccgttcgatattgtttgaaccgtggtcacactcctcttgccattgacaatggtttctctggcttcttgcgccatgattctattcatgaactttcatggattgatgttgatgagtggtgcattcgtggtggtagtgaaatcggtaccaaccgtgatacccctgatctcgacatgggcttcactgccttcaagttccaacaacataagattgatgctttgattattattggtggtttcgaggctttcaccgccctttctcaacttgagagtgcacgtgttaactacccttcattccgcattcccatggctatcatccctgccaccatttccaacaacgttcccggtaccgaattctctttgggttgcgatacttgtcttaatgccgttatggaatactgtgataccattaagcaaagtgctagtgctagccgtcgtcgtgtcttcgtttgtgaagttcaaggtggccgctctggttatatcgctactgtcggtggtctcattactggtgcctccgccatttacacccccgaggatggtatctccttggatatgttgcgtaaggatattgatcaccttaaggctacattcgccttagaagctggtcgtaaccgtgctggtcaacttatccttcgtaacgaatgtgcttctaaggtttacaccaccgaagttattggaaacatcatcagtgaagaagctcataagcgcttctccgctcgtaccgctgttcccggtcacgttcaacaaggtggtaaccccactcctatggaccgtgctcgtgctgctcgtctggctatgcgtgccattcgtttcttcgaaacttgccgtgccaacgacttgggtaatgaccccagctctgccgttgtcatcggtatccgtggtactggtgtctctttcagctctgttgctgatgttgagaacaacgaaaccgaaattgagatgcgtcgtcctaagaacgcttggtggcgtgatatgcacaacttggttaacatcttggccggcaagacctttgctgattaa

SEQ ID NO: 10 - protein, 942 aa, Schizosaccharomyces pombe
MSGETVHGISCYSVVANTEDTYNQTLDFYQKLGFKKVASFGTSDSDNARVCNESLREDWMHVAGNNSAESVTIKFRLVPGELSLSPAAEDSEWRGQKSSLVFYYPNLLDLLKQLSADAIKYQAFPNEKKPDEVYVEDPLGNLIGFSDRYNPFAHANLKKSEESGAASNLESGLATPVVETLKKATTSDKPAGVKKKIAVMTSGGDSPGMNAVVRAVARIAIHRGCDAFAIYEGYEGLVQGGDMIKQLQWGDVRGWLAEGGTLIGTARCMAFRERPGRLRAAKNLISAGIDSIIVCGGDGSLTGADIFRSDWPGLVKELEDTKAITPEQAKLYRHLTIVGLVGSIDNDMSSTDVTIGAFSSLHRICEAVDSISSTAISHSRAFIVEVMGRHCGWLAVLAALATGADFVFIPERPAEVGKWQDELCNSLSSVRKLGKRKSIVIVAEGAIDSELNHISPEDIKNLLVERLHLDTRVTTLGHVQRGGIPCAYDRMLATLQGVDAVDAVLASTPDTPSPMIAINGNKINRKPLMEAVKLTHEVADAIEKKQFAHAMELRDPEFADYLHTWEGTTFIEDESHFVPKDERMRVAIIHVGAPAGGMNSATRAAVRYCLNRGHTPLAIDNGFSGFLRHDSIHELSWIDVDEWCIRGGSEIGTNRDTPDLDMGFTAFKFQQHKIDALIIIGGFEAFTALSQLESARVNYPSFRIPMAIIPATISNNVPGTEFSLGCDTCLNAVMEYCDTIKQSASASRRRVFVCEVQGGRSGYIATVGGLITGASAIYTPEDGISLDMLRKDIDHLKATFALEAGRNRAGQLILRNECASKVYTTEVIGNIISEEAHKRFSARTAVPGHVQQGGNPTPMDRARAARLAMRAIRFFETCRANDLGNDPSSAVVIGIRGTGVSFSSVADVENNETEIEMRRPKNAWWRDMHNLVNILAGKTFAD

SEQ ID NO: 11 - DNA, 1215 bp, Propionibacterium freudenreichii
atggtgaaaaaggtcgctctgctgaccgctggtggcttcgccccctgtctttcctcggccatcgctgagctcatcaagcgctataccgaggtatcacccgaaacgaccctcatcggctatcgctatggctatgagggcctgctcaagggcgattccctcgagttctcccctgccgtgcgcgcacactacgaccggctcttcagcttcggcgggtcaccgatcgggaactcccgggtcaagctcaccaatgtgaaggacctggttgcgcggggcctggttgcttccggcgatgatcccctcaaggttgccgccgatcagctgattgccgacggggtcgacgtgctgcacacgatcgggggcgacgacaccaacaccacggccgccgacctggccgcctacctggcacagcatgactacccgctgacggttgtggggctgcccaagacgatcgacaacgacatcgtgcccatccgccagtcgctgggtgcctggacggccgccgacgagggggcccgcttcgcggcgaatgtgatcgccgagcacaatgctgctccgcgcgaactcatcatccacgagatcatgggccgcaactgcggctatctggcggccgagacctcgcggcgttacgtggcctggctcgacgcgcagcagtggctcccggaggccggtctcgaccgacgtggctgggatatccacgccctgtacgtgccggaggccacgatcgacctggacgccgaggccgagcgcctgcgcaccgtgatggacgaggtgggaagcgtcaatatcttcatctcggagggagccggcgttcccgatatcgtcgcccagatgcaggccacgggccaggaggtgcccactgacgccttcggccacgtgcagctcgacaagatcaatcccggggcgtggttcgccaagcagttcgccgagcgcatcggtgcgggcaagaccatggtgcagaagtccggttacttcagccgctccgccaagtcgaatgcccaggacctggagctcatcgccgccaccgccacgatggcggttgatgcggcgctggccggcacccccggcgtggtcggtcaggacgaggaggcaggcgacaagctgagcgtgatcgacttcaagcggatcgcgggccacaagcccttcgacatcacccttgattggtacacccagctgctggcccgaatcggtcagccggcacccatcgccgccgcgtaa

SEQ ID NO: 12 - protein, 404 aa, Propionibacterium freudenreichii
MVKKVALLTAGGFAPCLSSAIAELIKRYTEVSPETTLIGYRYGYEGLLKGDSLEFSPAVRAHYDRLFSFGGSPIGNSRVKLTNVKDLVARGLVASGDDPLKVAADQLIADGVDVLHTIGGDDTNTTAADLAAYLAQHDYPLTVVGLPKTIDNDIVPIRQSLGAWTAADEGARFAANVIAEHNAAPRELIIHEIMGRNCGYLAAETSRRYVAWLDAQQWLPEAGLDRRGWDIHALYVPEATIDLDAEAERLRTVMDEVGSVNIFISEGAGVPDIVAQMQATGQEVPTDAFGHVQLDKINPGAWFAKQFAERIGAGKTMVQKSGYFSRSAKSNAQDLELIAATATMAVDAALAGTPGVVGQDEEAGDKLSVIDFKRIAGHKPFDITLDWYTQLLARIGQPAPIAAA

SEQ ID NO: 13 - DNA, 927 bp, Escherichia coli
atggtacgtatctatacgttgacacttgcgccctctctcgatagcgcaacaattaccccgcaaatttatcccgaggaaaactgcgctgtaccgcaccggtgttcgaacccgggcggcggcatcaacgtcgcccgcgccattgcccatcttggaggcagtgccacagcgatcttcccggcgggtggcgcgaccggcgaacacctggtttcactgttggcggatgaaaatgtccccgtcgctactgtagaagccaaagactggacccggcagaatttacacgtacatgtggaagcaagcggtgagcagtatcgttttgttatgccaggcgcggcattaaatgaagatgagtttcgccagcttgaagagcaagttctggaaattgaatccggggccatcctggtcataagcggaagcctgccgccaggtgtgaagctggaaaaattaacccaactgatttcgctgcgcaaaaacaagggatccgctgcatcgtcgacagttcttggacagggcttaagtgcagcactggcaattggtaacatcgagttggttaagcctaaccaaaaagaactcagtgcgctggtgaatcgcgaactcacccagccggacgatgtccgcaaagccgcgcaggaaatcgttaatagcggcaaggccaaacgggttgtcgtttccctgggtccacaaggagcgctgggtgttgatagtgaaaactgtattcaggtggtgccaccagcgttgaaaagccagagtaccgttggcgctggtgacagactggtcggcgcgatgacactgaaactggcagaaaatgcctctcttgaagagatggttcgttttggcgtagctgcggggagtgcagccacactcaatcagggaacacgtctgtgctcccatgacgatacgcaaaaaatttacgcttacctttcccgctaa

SEQ ID NO: 14 - protein, 308 aa, Escherichia coli
MVRIYTLTLAPSLDSATITPQIYPEENCAVPHRCSNPGGGINVARAIAHLGGSATAIFPAGGATGEHLVSLLADENVPVATVEAKDWTRQNLHVHVEASGEQYRFVMPGAALNEDEFRQLEEQVLEIESGAILVISGSLPPGVKLEKLTQLISLRKNKGSAASSTVLGQGLSAALAIGNIELVKPNQKELSALVNRELTQPDDVRKAAQEIVNSGKAKRVVVSLGPQGALGVDSENCIQVVPPALKSQSTVGAGDRLVGAMTLKLAENASLEEMVRFGVAAGSAATLNQGTRLCSHDDTQKIYAYLSR

(19) United States
(12) Patent Application Publication
(10) Pub. No.: US 2006/0288451 A1
(43) Pub. Date: Dec. 21, 2006

(54) Elevation of oil in monocot plants

(75) Inventors: Dale Val, Zamora, CA (US); Dang Yang Ke, Sacramento, CA (US)

Correspondence Address: FULBRIGHT & JAWORSKI, LLP, 600 CONGRESS AVENUE, SUITE 2400, AUSTIN, TX, 78745, US

(73) Assignee: MONSANTO TECHNOLOGY, L.L.C.

(21) Appl. No.: 11/440,802

(22) Filed: May 25, 2006

Related U.S. Application Data
(60) Provisional application No. 60/684,809, filed on May 26, 2005.

(51) Int. Cl. A01H 5/00; C11B 1/00; C12N 15/82; C12N 5/04; A23K 1/165

(52) U.S. Cl. 800/281; 800/320; 800/320.1; 800/320.2; 800/320.3; 424/442; 554/8; 435/468; 435/412

(57) ABSTRACT

Methods of making crop plants having higher oil levels in their seeds by increasing glycolytic flux through over-expression of nucleic acids encoding phosphofructokinase are provided. The invention may further comprise the over-expression of nucleic acids encoding a pyruvate kinase to alter oil content in plant seeds, and monocot cells and plants transformed with phosphofructokinase, or phosphofructokinase and pyruvate kinase transgenes.

This application claims priority under 35 U.S.C. 119(e) from Provisional Application U.S. Ser. No. 60/684,809, filed May 26, 2005, which application is incorporated herein by reference.

### BACKGROUND OF THE INVENTION

1. Field of the Invention

This invention relates to increasing oil levels in the seeds of crop plants by over-expression of phosphofructokinase.

2. Related Art

The conversion of fructose-6-phosphate (F-6-P) to fructose-1,6-bis-phosphate (F-1,6-BP) is catalyzed by the enzyme phosphofructokinase (PFK). ATP-dependent PFK catalyzes this step in most organisms and tissues and this enzyme has long been implicated in the regulation of glycolytic flux. Indeed in many systems, including plants, the combined regulation of the allosteric enzymes ATP-PFK and pyruvate kinase (PK) is believed to be primarily responsible for regulating glycolysis. In plants, ATP-PFK is located in the plastids and the cytosol. Frequently the enzymes found in these different cellular locations have different kinetic properties. In addition to ATP-PFK enzymes, there are two other enzymes that are involved in the interconversion of these two metabolites: pyrophosphate-dependent PFK (PPI-PFK), which catalyzes the inorganic pyrophosphate-dependent reversible interconversion of F-6-P and F-1,6-BP, and fructose-1,6-bisphosphatase, which catalyzes the reverse reaction for gluconeogenesis.

Doehlert et al. (1988) found that PFK was more abundant in embryos (high oil tissue) than in endosperm (low oil tissue) of corn. In a survey of the distribution of the abundance of enzymes involved in carbohydrate metabolism within different parts of the kernel, these workers found that PFK activity correlated with those areas of the kernel that deposited the most oil. There is a large body of evidence supporting the importance of PFK in regulating glycolytic flux (e.g. Plaxton, 1996). Although some transgenic plants comprising a heterologous phosphofructokinase gene have been generated (e.g. U.S. Pat. No. 7,012,171; Burrell et al., 1994; Thomas et al., 1997; WO 99/67392; Wood et al., 1999; Wood et al., 2002), the use of PFK to increase oil content in monocot plants and seeds has not been reported.

In order to produce higher oil levels in developing seeds of monocots, these tissues need to convert more of the incoming carbon (predominantly sucrose) into triacylglycerols (TAG) rather than starch. This suggests that more of the hexoses need to be broken down by glycolysis in order to generate pyruvate and acetyl-CoA as substrates for fatty acid synthesis.

### SUMMARY OF THE INVENTION

This invention involves the over expression of a pfk gene with the intended effect of increased glycolytic flux and thus increased substrate supply, resulting in higher oil levels in tissues such as the seeds of monocot plants. More specifically it involves the over-expression of the ATP-dependent pfk gene from the bacteria Lactobacillus delbreuckii subspecies bulgaricus in the seeds of monocots.

This invention provides a method of making a monocot plant having increased oil in its seed, comprising the step of growing a transformed monocot plant comprising a nucleic acid sequence encoding a phosphofructokinase, operably linked to a seed-enhanced promoter which is also optionally operably linked to a nucleic acid sequence encoding a plastid transit peptide except when said seed-enhanced promoter is an embryo-enhanced promoter, to produce seed, whereby the oil content of the seed is increased as compared to a seed of an isogenic plant lacking the nucleic acid sequence.

This invention provides a method of making a monocot plant having increased oil in its seeds, comprising the step of growing a transformed monocot plant comprising a nucleic acid sequence encoding a phosphofructokinase other than SEQ ID NO:9 or 13, operably linked to a seed-enhanced promoter which is also optionally operably linked to a nucleic acid sequence encoding a plastid transit peptide except when said seed-enhanced promoter is an embryo-enhanced promoter, to produce seed, whereby the oil content of the seed is increased as compared to a seed of an isogenic plant lacking the nucleic acid sequence.

In one embodiment, the method comprises making a monocot plant wherein the nucleic acid sequence encoding a phosphofructokinase is selected from the group consisting of:

a) nucleic acid sequences comprising SEQ ID NO:1 or 11 and

b) nucleic acid sequences encoding SEQ ID NO:2 or 12.

In another embodiment, the plant further comprises a second nucleic acid sequence encoding a pyruvate kinase, operably linked to a seed-enhanced promoter. In one version of this embodiment, the second nucleic acid sequence encoding a pyruvate kinase is selected from the group consisting of:

a) a nucleic acid sequence comprising SEQ ID NO:3 and

b) a nucleic acid sequence encoding SEQ ID NO:4.

In various embodiments, the monocot plant is selected from the group consisting of corn (Zea mays), rice (Oryza sativa), barley (Hordeum vulgare), millet (Panicum miliaceum), rye (Secale cereale), wheat (Triticum aestivum), and sorghum (Sorghum bicolor).

In various embodiments, the promoter is selected from the group consisting of embryo-enhanced promoters, endosperm-enhanced promoters and embryo- and endosperm-enhanced promoters.

The invention also provides transformed plant cells, transformed plants and progeny, seed, oil and meal. Additionally, the invention provides animal feed and human food compositions and methods of producing oil.

### BRIEF DESCRIPTION OF THE SEQUENCES

SEQ ID NO:1 sets forth a nucleic acid sequence encoding a phosphofructokinase from Lactobacillus delbreuckii ssp. bulgaricus.

SEQ ID NO:2 sets forth a polypeptide sequence of a phosphofructokinase from Lactobacillus delbreuckii ssp. bulgaricus.

SEQ ID NO:3 sets forth a nucleic acid sequence encoding a pyruvate kinase from Lactobacillus delbreuckii ssp. bulgaricus.

SEQ ID NO:4 sets forth a polypeptide sequence of a pyruvate kinase from Lactobacillus delbreuckii ssp. bulgaricus.

SEQ ID NOs: 5-8 set forth nucleic acid primers.

SEQ ID NO:9 sets forth a nucleic acid sequence encoding a phosphofructokinase from Schizosaccharomyces pombe.

SEQ ID NO:10 sets forth a polypeptide sequence of a phosphofructokinase from Schizosaccharomyces pombe.

SEQ ID NO:11 sets for a nucleic acid sequence encoding a phosphofructokinase from Propionibacterium freudenreichii.

SEQ ID NO:12 sets forth a polypeptide sequence of a phosphofructokinase from Propionibacterium freudenreichii.

SEQ ID NO:13 sets forth a nucleic acid sequence encoding a phosphofructokinase from Escherichia coli.

SEQ ID NO:14 sets forth a polypeptide sequence of a phosphofructokinase from Escherichia coli.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows an alignment of the coding sequence of the pfk gene (SEQ ID NO:1) isolated from Lactobacillus delbreuckii subspecies bulgaricus ATCC strain 11842 with the published pfk gene sequence (EMBL accession # X71403).

FIG. 2 depicts plasmid pMON72008.

FIG. 3 depicts plasmid pMON79823.

FIG. 4 depicts plasmid pMON79824.

FIG. 5 depicts plasmid pMON79827.

FIG. 6 depicts plasmid pMON72028.

FIG. 7 depicts plasmid pMON79832.

FIG. 8 depicts plasmid pMON81470.

FIG. 9 depicts plasmid pMON72029.

FIG. 10 depicts plasmid pMON83715.

### DESCRIPTION OF ILLUSTRATIVE EMBODIMENTS

The following definitions are provided as an aid to understanding this invention. The phrases “DNA sequence,” “nucleic acid sequence,” “nucleic acid molecule,” and “nucleic acid segment” refer to a physical structure comprising an orderly arrangement of nucleotides. The DNA segment, sequence, or nucleotide sequence may be contained within a larger nucleotide molecule, vector, or the like. In addition, the orderly arrangement of nucleic acids in these sequences may be depicted in the form of a sequence listing, figure, table, electronic medium, or the like.

The phrases “coding sequence,” “coding region,” “structural sequence,” and “structural nucleic acid sequence” refer to all or a segment of a DNA sequence, nucleic acid sequence, nucleic acid molecule in which the nucleotides are arranged in a series of triplets that each form a codon. Each codon encodes a specific amino acid. Thus, the coding sequence, coding region, structural sequence, and structural nucleic acid sequence encode a series of amino acids forming a protein, polypeptide, or peptide sequence. The coding sequence, coding region, structural sequence, and structural nucleic acid sequence may be contained within a larger nucleic acid molecule, vector, or the like. In addition, the arrangement of nucleotides in these sequences may be depicted in the form of a sequence listing, figure, table, electronic medium, or the like.

The term “cDNA” refers to a double-stranded DNA that is complementary to and derived from mRNA.

“Expression” refers to the process by which a gene's coded information is converted into structures present and operating in the cell. Expressed genes include those that are transcribed into RNA and then translated into protein and those that are transcribed into RNA but not translated into protein (e.g., transfer RNA and ribosomal RNA).

As used herein, “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. An “exogenous” gene or “transgene” refer to a gene that has been introduced into the genome by a transformation procedure. A transgene includes genomic DNA introduced by a transformation procedure (e.g., a genomic DNA linked to its active promoter).

“Heterologous” refers to the relationship between two or more nucleic acid or protein sequences that are derived from different sources. For example, a promoter is heterologous with respect to a coding sequence if such a combination is not normally found in nature. In addition, a particular nucleic acid sequence may be “heterologous” with respect to a cell or organism into which it is inserted if it does not naturally occur in that particular cell or organism.

“Sequence homology” refers to the level of similarity between 2 or more nucleic acid or amino acid sequences in terms of percent of positional identity. The term homology is also used to refer to the concept of similar functional properties among different nucleic acids or proteins.

“Hybridization” refers to the ability of a first strand of nucleic acid to join with a second strand via hydrogen bond base pairing when the two nucleic acid strands have sufficient sequence complementarity. As used herein, a nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Thus two nucleic acid strands are said to have sufficient complementarity when they can hybridize to one another with sufficient stability to permit them to remain annealed to one another under appropriate conditions.

Appropriate stringency conditions which promote DNA hybridization are, for example, 6.0× sodium chloride/sodium citrate (SSC) at about 45° C., followed by a wash of 2.0×SSC at 20-25° C., and are known to those skilled in the art. For example, the salt concentration in the wash step can be selected from a low stringency of about 2.0×SSC at 50° C. to a high stringency of about 0.2×SSC at 65° C. In addition, the temperature in the wash step can be increased from low stringency conditions at room temperature, about 22° C., to high stringency conditions at about 65° C. Both temperature and salt may be varied, or either the temperature or the salt concentration may be held constant such that a nucleic acid will specifically hybridize to one or more of the polynucleotide molecules provided herein, for example, as set forth in: SEQ ID NOs 1, 3, or 11, and complements thereof, under moderately stringent conditions, for example at about 2.0×SSC and about 65° C.

The phrase “isolated” means having been removed from its natural environment, regardless of its eventual disposition. For example, a nucleic acid sequence “isolated” from rice, such as by cloning from a rice cell, remains “isolated” when it is inserted into the genome of a corn cell.

The phrase “operably linked” refers to the spatial arrangement of two or more nucleic acid regions or nucleic acid sequences so that they exert their appropriate effects with respect to each other. For example, a promoter region may be positioned relative to a nucleic acid sequence such that transcription of the nucleic acid sequence is directed by the promoter region. The promoter region and the nucleic acid sequence are “operably linked.”

The term “phosphofructokinase” refers to an enzyme capable of converting fructose-6-phosphate (F-6-P) to fructose-1,6-bis-phosphate (F-1,6-BP). This includes enzymes from the International Union of Biochemistry and Molecular Biology Enzyme Nomenclature classes EC 2.7.1.1 and EC 2.7.1.90.

The term “pyruvate kinase” refers to an enzyme capable of converting phosphoenol pyruvate to pyruvate. This includes enzymes from the International Union of Biochemistry and Molecular Biology Enzyme Nomenclature class EC 2.7.1.40.

The term “plastid” refers to a self-replicating cytoplasmic organelle of algal and plant cells, such as a chloroplast or chromoplast. A “transit peptide” refers to a sequence of amino acids at the N-terminus of a protein that targets the polypeptide to the plastid from its synthesis in the cytosol and facilitates its translocation through the plastid membrane. After the polypeptide enters the plastid, the transit peptide is cleaved from the polypeptide.

“Upstream” and “downstream” are positional terms used with reference to the location of a nucleotide sequence and the direction of transcription or translation of coding sequences, which normally proceeds in the 5′ to 3′ direction.

The terms “promoter” or “promoter region” refer to a nucleic acid sequence, usually found upstream (5′) to a coding sequence, that is capable of directing transcription of a nucleic acid sequence into an RNA molecule. The promoter or promoter region typically provides a recognition site for RNA polymerase and the other factors necessary for proper initiation of transcription. As contemplated herein, a promoter or promoter region includes variations of promoters derived by inserting or deleting regulatory regions, subjecting the promoter to random or site-directed mutagenesis, and the like. The activity or strength of a promoter may be measured in terms of the amounts of RNA it produces, or the amount of protein accumulation in a cell or tissue, relative to a second promoter that is similarly measured.

The phrase “3′ non-coding sequences” refers to nucleotide sequences located downstream of a coding sequence and include polyadenylation recognition sequences and other sequences encoding regulatory signals capable of affecting mRNA processing or gene expression. These are commonly referred to as 3′-untranslated regions or 3′-UTRs. The polyadenylation signal is usually characterized by affecting the addition of polyadenylic acid tracts to the 3′ end of the mRNA precursor. The use of different 3′ non-coding sequences is exemplified by Ingelbrecht et al. (1989).

“Translation leader sequence” or “5′-untranslated region” or “5′-UTR” all refer to a nucleotide sequence located between the promoter sequence of a gene and the coding sequence. The 5′-UTR is present in the fully processed mRNA upstream of the translation start sequence. The 5′-UTR may affect processing of the primary transcript to mRNA, mRNA stability or translation efficiency. Examples of translation leader sequences have been described (Turner and Foster, 1995).

“RNA transcript” refers to the product resulting from RNA polymerase-catalyzed transcription of a DNA sequence. When the RNA transcript is a perfect complementary copy of the DNA sequence, it is referred to as the primary transcript. An RNA sequence derived from posttranscriptional processing of the primary transcript is referred to as the mature RNA. “Messenger RNA” (mRNA) refers to the RNA that is without introns and that can be translated into polypeptide by the cell.

“Recombinant vector” refers to any agent by or in which a nucleic acid of interest is amplified, expressed, or stored, such as a plasmid, cosmid, virus, autonomously replicating sequence, phage, or linear single-stranded, circular single-stranded, linear double-stranded, or circular double-stranded DNA or RNA nucleotide sequence. The recombinant vector may be synthesized or derived from any source and is capable of genomic integration or autonomous replication.

“Regulatory sequence” refers to a nucleotide sequence located upstream (5′), within, or downstream (3′) with respect to a coding sequence, or an intron, whose presence or absence affects transcription and expression of the coding sequence

“Substantially homologous” refers to two sequences that are at least about 90% identical in sequence, as measured by the CLUSTAL W algorithm in, for example DNAStar (Madison, Wis.).

“Substantially purified” refers to a molecule separated from substantially all other molecules normally associated with it in its native state. More preferably, a substantially purified molecule is the predominant species present in a preparation. A substantially purified molecule may be greater than about 60% free, preferably about 75% free, more preferably about 90% free, and most preferably about 95% free from the other molecules (exclusive of solvent) present in the natural mixture. The phrase “substantially purified” is not intended to encompass molecules present in their native state. Preferably, the nucleic acid molecules and polypeptides of this invention are substantially purified.

The term “transformation” refers to the introduction of nucleic acid into a recipient host. The term “host” refers to bacteria cells, fungi, animals or animal cells, plants or seeds, or any plant parts or tissues including plant cells, protoplasts, calli, roots, tubers, seeds, stems, leaves, seedlings, embryos, and pollen.

As used herein, a “transgenic plant” is a plant having stably introduced into its genome, for example, the nuclear or plastid genomes, an exogenous nucleic acid.

The term “isogenic” as a comparative term between plants or plant lines having or lacking a transgene means plants or lines having the same or similar genetic backgrounds, with the exception of the transgene in question. For example, so-called sister lines representing phenotypically similar or identical selections from the same parent F₂ population are considered to be “isogenic.” When the progeny of a stable transformant plant are crossed and backcrossed with the plants of the untransformed parent line for 3 to 6 generations (or more) using the untransformed parent as the recurrent parent while selecting for type (genotype by molecular marker analysis, phenotype by field observation, or both) and for the transgene, the resulting transgenic line is considered to be highly “isogenic” to its untransformed parent line.

The terms “seeds” “kernels” and “grain” are understood to be equivalent in meaning. The term kernel is frequently used in describing the seed of a corn or rice plant. In all plants the seed is the mature ovule consisting of a seed coat, embryo, aleurone, and an endosperm.

Nucleic Acids Encoding Phosphofructokinase and Pyruvate Kinase

This invention provides, among other things, a method of using nucleic acid molecules encoding phosphofructokinase (International Union of Biochemistry and Molecular Biology Enzyme Nomenclature classes EC 2.7.1.11 and EC 2.7.1.90; more specifically SEQ ID NOs: 1 and 11) and pyruvate kinase (EC 2.7.1.40; more specifically SEQ ID NO:3).

In one embodiment, these nucleic acid molecules are used in the context of this invention for altering the oil content of a seed in a monocot plant.

Such nucleic acid molecules can be amplified using cDNA, mRNA or genomic DNA as a template and appropriate oligonucleotide primers according to standard PCR™ amplification techniques. Alternatively, they can be synthesized using standard synthetic techniques, such as an automated DNA synthesizer.

If desired, the sequences of nucleic acids that code for phosphofructokinase or pyruvate kinase can be modified without changing the resulting amino acid sequence of the expressed protein so that the sequences are more amenable to expression in plant hosts. A coding sequence can be an artificial DNA. An artificial DNA, as used herein means a DNA polynucleotide molecule that is non-naturally occurring. Artificial DNA molecules can be designed by a variety of methods, such as, methods known in the art that are based upon substituting the codon(s) of a first polynucleotide to create an equivalent, or even an improved, second-generation artificial polynucleotide, where this new artificial polynucleotide is useful for enhanced expression in transgenic plants. The design aspect often employs a codon usage table, the table is produced by compiling the frequency of occurrence of codons in a collection of coding sequences isolated from a plant, plant type, family or genus. Other design aspects include reducing the occurrence of polyadenylation signals, intron splice sites, or long AT or GC stretches of sequence (U.S. Pat. No. 5,500,365). Full length coding sequences or fragments thereof can be made of artificial DNA using methods known to those skilled in the art.

Expression Vectors and Cassettes

A plant expression vector can comprise a native or normative promoter operably linked to an above-described nucleic acid molecule. The selection of promoters, e.g., promoters that may be described as strongly expressed, weakly expressed, inducibly expressed, tissue-enhanced expressed (i.e., specifically or preferentially expressed in a tissue), organ-enhanced expressed (i.e., specifically or preferentially expressed in an organ) and developmentally-enhanced expressed (i.e., specifically Pr preferentially expressed during a particular stage(s) of development), is within the skill in the art. Similarly, the combining of a nucleic acid molecule as described above with a promoter is also within the skill in the art (see, e.g., Sambrook et al., 1989).

In one embodiment of this invention, an above-described nucleic acid molecule is operably linked to a seed-enhanced promoter causing expression sufficient to increase oil in the seed of a monocot plant. Promoters of the instant invention generally include, but are not limited to, promoters that function in bacteria, bacteriophages, or plant cells. Useful promoters for bacterial expression are the lacZ, Sp6, T7, T5 or E. coli glgC promoters. Useful promoters for plants cells include the globulin promoter (see for example Belanger and Kriz (1991), gamma zein Z27 promoter (see, for example, Lopes et al. (1995), L3 oleosin promoter (U.S. Pat. No. 6,433,252), barley PER1 promoter (Stacey et al. (1996), CaMV 35S promoter (Odell et al. (1985)), the CaMV 19S (Lawton et al., 1987), nos (Ebert et al., 1987), Adh (Walker et al., 1987), sucrose synthase (Yang et al., 1990), actin (Wang et al., 1992), cab (Sullivan et al., 1989), PEPCase promoter (Hudspeth et al., 1989), or those associated with the R gene complex (Chandler et al., 1989). The Figwort Mosaic Virus (FMV) promoter (Richins et al., 1987), arcelin, tomato E8, patatin, ubiquitin, mannopine synthase (mas) and tubulin promoters are other examples of useful promoters.

Promoters expressed in maize include promoters from genes encoding zeins, which are a group of storage proteins found in maize endosperm. Genomic clones for zein genes have been isolated (Pedersen et al., 1982) and Russell et al., 1997) and the promoters from these clones, including the 15 kD, 16 kD, 19 kD, 22 kD, and 27 kD genes, can be used. Other seed-expression enhanced promoters known to function in maize and in other plants include the promoters for the following genes: Waxy (granule bound starch synthase), Brittle and Shrunken 2 (ADP glucose pyrophosphorylase), Shrunken 1 (sucrose synthase), branching enzymes I and II, starch synthases, debranching enzymes, oleosins, glutelins, and Betl1 (basal endosperm transfer layer). Other promoters useful in the practice of the invention that are known by one of skill in the art are also contemplated by the invention.

Moreover, transcription enhancers or duplications of enhancers can be used to increase expression from a particular promoter. Examples of such enhancers include, but are not limited to the Adh intron1 (Callis et al., 1987), a rice actin intron (McElroy et al., 1991; U.S. Pat. No. 5,641,876), sucrose synthase intron (Vasil et al., 1989), a maize HSP70 intron (also referred to as Zm.DnaK) (U.S. Pat. No. 5,424,412 Brown, et al.)) a TMV omega element (Gallie et al., 1999), the CaMV 35S enhancer (U.S. Pat. Nos. 5,359,142 & 5,196,525, McPherson et al.) or an octopine synthase enhancer (U.S. Pat. No. 5,290,924, Last et al.). As the DNA sequence between the transcription initiation site and the start of the coding sequence, i.e., the untranslated leader sequence, can influence gene expression, one may also wish to employ a particular leader sequence. Any leader sequence available to one of skill in the art may be employed. Preferred leader sequences direct optimum levels of expression of the attached gene, for example, by increasing or maintaining mRNA stability and/or by preventing inappropriate initiation of translation (Joshi, 1987). The choice of such sequences is at the discretion of those of skill in the art. Sequences that are derived from genes that are highly expressed in corn, rice and monocots in particular, are contemplated.

Expression cassettes of this invention will also include a sequence near the 3′ end of the cassette that acts as a signal to terminate transcription from a heterologous nucleic acid and that directs polyadenylation of the resultant mRNA. These are commonly referred to as 3′ untranslated regions or 3′ UTRs. Some 3′ elements that can act as transcription termination signals include those from the nopaline synthase gene of Agrobacterium tumefaciens (Bevan et al., 1983), a napin 3′ untranslated region (Kridl et al., 1991), a globulin 3′ untranslated region (Belanger and Kriz, 1991) or one from a zein gene, such as Z27 (Lopes et al., 1995). Other 3′ regulatory elements known to the art also can be used in the vectors of the invention.

Expression vectors of this invention may also include a sequence coding for a transit peptide fused to the heterologous nucleic acid sequence. Chloroplast transit peptides (CTPs) are engineered to be fused to the N-terminus of a protein to direct the protein into the plant chloroplast. Many chloroplast-localized proteins are expressed from nuclear genes as precursors and are targeted to the chloroplast by a chloroplast transit peptide that is removed during the import process. Examples of other such chloroplast proteins include the small subunit (SSU) of Ribulose-1,5-bisphosphate carboxylase, ferredoxin, ferredoxin oxidoreductase, the light-harvesting complex protein I and protein II, and thioredoxin F. In particular, the CTP of the Nicotiana tabacum ribulose 1,5-bisphosphate carboxylase small subunit choroplast transit peptide (SSU-CTP) (Mazur, et al., 1985) could be used. It has been demonstrated in vivo and in vitro that non-chloroplast proteins may be targeted to the chloroplast by use of protein fusions with a CTP and that a CTP sequence is sufficient to target a protein to the chloroplast. Incorporation of a suitable chloroplast transit peptide, such as, the Arabidopsis thaliana EPSPS CTP (Klee et al., 1987), and the Petunia hybrida EPSPS CTP (della-Cioppa et al., 1986) has been shown to target heterologous EPSPS protein sequences to chloroplasts in transgenic plants.

This invention further provides a vector comprising an above-described nucleic acid molecule. A nucleic acid molecule as described above can be cloned into any suitable vector and can be used to transform or transfect any suitable host. The selection of vectors and methods to construct them are commonly known to the art and are described in general technical references (see, in general, “Recombinant DNA Part D” (1987)). The vector will preferably comprise regulatory sequences, such as transcription and translation initiation and termination codons, which are specific to the type of host (e.g., bacterium, fungus, or plant) into which the vector is to be introduced, as appropriate and taking into consideration whether the vector is DNA or RNA.

Constructs of vectors that are circular or linear can be prepared to contain an entire nucleic acid sequence as described above or a portion thereof ligated to a replication system functional in a prokaryotic or eukaryotic host cell. Replication systems can be derived from ColE1, 2 mμ plasmid, λ phage, f1 filamentous phage, Agrobacterium species (e.g., A. tumefaciens and A. rhizogenes), and the like.

In addition to the replication system and the inserted nucleic acid sequence, the construct can include one or more marker genes that allow for selection of transformed or transfected hosts. Marker genes include biocide resistance, such as resistance to antibiotics, heavy metals, herbicides, etc., complementation in an auxotrophic host to provide prototrophy, and the like.

This invention provides a host cell comprising an above-described nucleic acid molecule, optionally in the form of a vector. Suitable hosts include plant, bacterial and yeast cells, including Escherichia coli, Bacillus subtilis, Agrobacterium tumefaciens, Saccharomyces cerevisiae, and Neurospora crassa. E. coli hosts include TB-1, TG-2, DH5α, XL-Blue MRF′ (Stratagene, La Jolla, Calif.), SA2821, Y1090 and TG02. Plant cells include cells of monocots, including, but not limited to corn, wheat, barley, oats, rye, millet, sorghum, and rice.

Polypeptides

This invention provides phosphofructokinases and, in some instances, a pyruvate kinase encoded by an above-described nucleic acid molecule. The polypeptide preferably comprises an amino end and a carboxyl end. The polypeptide can comprise D-amino acids, L-amino acids or a mixture of D- and L-amino acids.

Alterations of the native amino acid sequence to produce variant polypeptides can be done by a variety of means known to those ordinarily skilled in the art. For instance, amino acid substitutions can be conveniently introduced into the polypeptides by changing the sequence of the nucleic acid molecule at the time of synthesis. Site-specific mutations can also be introduced by ligating into an expression vector a synthesized oligonucleotide comprising the modified sequence. Alternately, oligonucleotide-directed, site-specific mutagenesis procedures can be used, such as disclosed in Walder et al. (1986); Bauer et al. (1985); and U.S. Pat. Nos. 4,518,584 and 4,737,462.

It is within the skill of the ordinary artisan to select synthetic and naturally-occurring amino acids that effect conservative or neutral substitutions for any particular naturally-occurring amino acids. The ordinarily skilled artisan desirably will consider the context in which any particular amino acid substitution is made, in addition to considering the hydrophobicity or polarity of the side-chain, the general size of the side chain and the pK value of side-chains with acidic or basic character under physiological conditions. For example, lysine, arginine, and histidine are often suitably substituted for each other, and more often arginine and histidine. As is known in the art, this is because all three amino acids have basic side chains, whereas the pK value for the side-chains of lysine and arginine are much closer to each other (about 10 and 12) than to histidine (about 6). Similarly, glycine, alanine, valine, leucine, and isoleucine are often suitably substituted for each other, with the proviso that glycine is frequently not suitably substituted for the other members of the group. This is because each of these amino acids is relatively hydrophobic when incorporated into a polypeptide, but glycine's lack of an α-carbon allows the phi and psi angles of rotation (around the α-carbon) so much conformational freedom that glycinyl residues can trigger changes in conformation or secondary structure that do not often occur when the other amino acids are substituted for each other. Other groups of amino acids frequently suitably substituted for each other include, but are not limited to, the group consisting of glutamic and aspartic acids; the group consisting of phenylalanine, tyrosine and tryptophan; and the group consisting of serine, threonine and, optionally, tyrosine. Additionally, the ordinarily skilled artisan can readily group synthetic amino acids with naturally-occurring amino acids.

If desired, the polypeptides can be modified, for instance, by glycosylation, amidation, carboxylation, or phosphorylation, or by the creation of acid addition salts, amides, esters, in particular C-terminal esters, and N-acyl derivatives of the polypeptides of the invention. The polypeptides also can be modified to create protein derivatives by forming covalent or noncovalent complexes with other moieties in accordance with methods known in the art. Covalently-bound complexes can be prepared by linking the chemical moieties to functional groups on the side chains of amino acids comprising the polypeptides, or at the N- or C-terminus. Desirably, such modifications and conjugations do not adversely affect the activity of the polypeptides (and variants thereof). While such modifications and conjugations can have greater or lesser activity, the activity desirably is not negated and is characteristic of the unaltered polypeptide.

The polypeptides (and fragments, variants and fusion proteins) can be prepared by any of a number of conventional techniques. The polypeptide can be isolated or substantially purified from a naturally occurring source or from a recombinant source. For instance, in the case of recombinant proteins, a DNA fragment encoding a desired protein can be subcloned into an appropriate vector using well-known molecular genetic techniques (see, e.g., Maniatis et al., 1989) and other references cited herein under “EXAMPLES”). The fragment can be transcribed and the protein subsequently translated in vitro. Commercially available kits also can be employed (e.g., such as manufactured by Clontech, Amersham Life Sciences, Inc., Arlington Heights, Ill.; Invitrogen, and the like). The polymerase chain reaction optionally can be employed in the manipulation of nucleic acids.

Such polypeptides also can be synthesized using an automated peptide synthesizer in accordance with methods known in the art. Alternately, the polypeptide (and fragments, variants, and fusion proteins) can be synthesized using standard peptide synthesizing techniques well-known to those of ordinary skill in the art (e.g., as summarized in Bodanszky, 1984)). In particular, the polypeptide can be synthesized using the procedure of solid-phase synthesis (see, e.g., Merrifield, 1963; Barany et al., 1987; and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the protein from the resin can be accomplished by, for example, acid treatment at reduced temperature. The polypeptide-containing mixture then can be extracted, for instance, with diethyl ether, to remove non-peptidic organic compounds, and the synthesized protein can be extracted from the resin powder (e.g., with about 25% w/v acetic acid). Following the synthesis of the polypeptide, further purification (e.g., using HPLC) optionally can be done in order to eliminate any incomplete proteins, polypeptides, peptides or free amino acids. Amino acid and/or HPLC analysis can be performed on the synthesized polypeptide to validate its identity. For other applications according to the invention, it may be preferable to produce the polypeptide as part of a larger fusion protein, either by chemical conjugation, or through genetic means known to the art. In this regard, this invention also provides a fusion protein comprising the polypeptide (or fragment thereof) or variant thereof and one or more other polypeptides/protein(s) having any desired properties or effector functions.

Assays for the production and identification of specific proteins are based on various physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches can be used to achieve even greater specificity such as western blotting in which antibodies are used to locate individual gene products that have been separated by electrophoretic techniques. Additional techniques can be used to absolutely confirm the identity of the product of interest such as evaluation by amino acid sequencing following purification. Although these are among the most common, other procedures can also be used.

Assay procedures can identify the expression of proteins by their functionality, particularly where the expressed protein is an enzyme capable of catalyzing chemical reactions involving specific substrates and products. For example, in plant extracts these reactions can be measured by providing and quantifying the loss of substrates or the generation of products of the reactions by physical and/or chemical procedures.

The activity of phosphofructokinase or pyruvate kinase can be measured in vitro using such an assay. Examples of such assays include LeBras et al. (1991) and LeBras et al. (1993). Metabolic radiotracer studies can measure the generation of different product pools in vivo. In such studies, radioactively labeled precursors are provided to intact tissues and the fate of the radioactive label is monitored as the precursor is metabolized.

In many cases, the expression of a gene product is determined by evaluating the phenotypic results of its expression. Such evaluations may be simply as visual observations, or may involve assays. Such assays can take many forms, such as analyzing changes in the chemical composition, morphology, or physiological properties of the plant. Chemical composition may be altered by expression of genes encoding enzymes or storage proteins that change amino acid composition and these changes can be detected by amino acid analysis, or by enzymes that change starch quantity, which can be analyzed by near infrared reflectance spectrometry. Morphological changes may include greater stature or thicker stalks.

The nucleic acid molecules, vectors and polypeptides of this invention can be used in agricultural methods and various screening assays. For example, a nucleic acid molecule can be used to express phosphofructokinase via a vector in a host cell, to detect mRNA encoding phosphofructokinase in a biological sample, to detect a genetic alteration in a gene encoding phosphofructokinase via a Southern blot, to suppress phosphofructokinase, or to up-regulate phosphofructokinase. The polypeptides can be used to compensate for deficiencies in phosphofructokinase or for the presence of a mutated phosphofructokinase having reduced or no activity in a plant, or to treat excessive levels of substrates, whether direct or indirect, for phosphofructokinase in a plant. Alternatively, the polypeptides can be used to screen agents for the ability to modulate their activity. The antibodies can be used to detect and isolate the respective polypeptides as well as decrease the availability of such polypeptides in vivo.

Methods

This invention provides a method of increasing oil in a seed of a monocot as compared to a seed of an untransformed plant having a similar genetic background. In one embodiment, the method of increasing oil comprises the step of growing a transformed monocot plant with a nucleic acid sequence encoding a phosphofructokinase other than SEQ ID NO:9 or 13 operably linked to a seed-enhanced promoter which is optionally operably linked to a nucleic acid sequence encoding a plastid transit peptide except when the seed-enhanced promoter is an embryo-enhanced promoter, to produce seed.

In another embodiment, the method of increasing oil comprises the step of introducing into cells of the monocot a nucleic acid sequence encoding a phosphofructokinase selected from the group consisting of:

a) nucleic acid sequences comprising SEQ ID NO:1 or 11 and

b) nucleic acid sequences encoding SEQ ID NO:2 or 12.

In another embodiment, the method of increasing oil comprises the further step of transforming the plant with a second nucleic acid sequence encoding a pyruvate kinase, operably linked to a seed-enhanced promoter. In yet another embodiment, the method of increasing oil comprises the further step of introducing into a plant a second nucleic acid sequence encoding a pyruvate kinase, selected from the group consisting of:

a) a nucleic acid sequence comprising SEQ ID NO:3 and

b) a nucleic acid sequence encoding SEQ ID NO:4.

In various embodiments, the monocot plant is selected from the group consisting of corn (Zea mays), rice (Oryza sativa), barley (Hordeum vulgare), millet (Panicum miliaceum), rye (Secale cereale), wheat (Triticum aestivum), and sorghum (Sorghum bicolor).

In various embodiments, the promoter is selected from the group consisting of embryo-enhanced promoters, endosperm-enhanced promoters and embryo- and endosperm-enhanced promoters.

Plant Transformation

In one embodiment of the invention, a transgenic plant expressing the desired protein or proteins is produced. Various methods for the introduction of a desired polynucleotide sequence encoding the desired protein into plant cells are known to the art, including: (1) physical methods such as microinjection, electroporation, and microparticle-mediated delivery (biolistics or gene gun technology); (2) virus-mediated delivery; and (3) Agrobacterium-mediated transformation.

The most commonly used methods for transformation of plant cells are the Agrobacterium-mediated DNA transfer process and the biolistics or microprojectile microparticle bombardment mediated process. Typically, nuclear transformation is desired but where it is desirable to specifically transform plastids, such as chloroplasts or amyloplasts, plant plastids may be transformed utilizing a microparticle-mediated delivery of the desired polynucleotide.

Agrobacterium-mediated transformation is achieved through the use of a genetically engineered soil bacterium belonging to the genus Agrobacterium. A number of wild-type and disarmed strains of Agrobacterium tumefaciens and Agrobacterium rhizogenes harboring Ti or Ri plasmids can be used for gene transfer into plants. Gene transfer is done via the transfer of a specific DNA known as “T-DNA” that can be genetically engineered to carry any desired piece of DNA into many plant species, as further elaborated, for example, in U.S. Pat. No. 6,265,638 to Bidney et al., the disclosures of which are hereby incorporated herein by reference.

Agrobacterium-mediated genetic transformation of plants involves several steps. The first step, in which the virulent Agrobacterium and plant cells are first brought into contact with each other, is generally called “inoculation”. Inoculation is preferably accompanied by some method of injury to some of the plant cells, which releases plant cellular constituents, such as coumaryl alcohol, sinapinate (which is reduced to acetosyringone), sinapyl alcohol, and coniferyl alcohol, that activate virulence factors in the Agrobacterium. Following the inoculation, the Agrobacterium and plant cells/tissues are permitted to grow together for a period of several hours to several days or more under conditions suitable for growth and T-DNA transfer. This step is termed “co-culture”, Following co-culture and T-DNA delivery, the plant cells are treated with bactericidal or bacteriostatic agents to kill the Agrobacterium remaining in contact with the explant and/or in the vessel containing the explant. If this is done in the absence of any selective agents to promote preferential growth of transgenic versus non-transgenic plant cells, then this is typically referred to as the “delay” step. If done in the presence of selective pressure favoring transgenic plant cells, then it is referred to as a “selection” step. When a “delay” is used, it is typically followed by one or more “selection” steps.

With respect to microparticle bombardment (U.S. Pat. No. 5,550,318 (Adams et al.); U.S. Pat. No. 5,538,880 (Lundquist et. al.), U.S. Pat. No. 5,610,042 (Chang et al.); and PCT Publication WO 95/06128 (Adams et al.); each of which is specifically incorporated herein by reference in its entirety), microscopic particles are coated with nucleic acids and delivered into cells by a propelling force. Exemplary particles include those comprised of tungsten, platinum, and preferably, gold.

An illustrative embodiment of a method for delivering DNA into plant cells by acceleration is the Biolistics Particle Delivery System (BioRad, Hercules, Calif.), which can be used to propel particles coated with DNA or cells through a screen, such as a stainless steel or Nytex screen, onto a filter surface covered with monocot plant cells cultured in suspension.

Microparticle bombardment techniques are widely applicable, and may be used to transform virtually any plant species. Examples of species that have been transformed by microparticle bombardment include monocot species such as maize (International Publication No. WO 95/06128 (Adams et al.)), barley, wheat (U.S. Pat. No. 5,563,055 (Townsend et al.) incorporated herein by reference in its entirety), rice, oat, rye, sugarcane, and sorghum; as well as a number of dicots including tobacco, soybean (U.S. Pat. No. 5,322,783 (Tomes et al.), incorporated herein by reference in its entirety), sunflower, peanut, cotton, tomato, and legumes in general (U.S. Pat. No. 5,563,055 (Townsend et al.) incorporated herein by reference in its entirety).

To select or score for transformed plant cells regardless of transformation methodology, the DNA introduced into the cell contains a gene that functions in a regenerable plant tissue to produce a compound that confers upon the plant tissue resistance to an otherwise toxic compound. Genes of interest for use as a selectable, screenable, or scorable marker would include but are not limited to beta-glucuronidase (GUS), green fluorescent protein (GFP), luciferase (LUX), antibiotic or herbicide tolerance genes. Examples of antibiotic resistance genes include the penicillins, kanamycin (and neomycin, G418, bleomycin); methotrexate (and trimethoprim); chloramphenicol; kanamycin and tetracycline. Polynucleotide molecules encoding proteins involved in herbicide tolerance are known in the art, and include, but are not limited to a polynucleotide molecule encoding 5-enolpyruvylshikimate-3-phosphate synthase (EPSPS) described in U.S. Pat. No. 5,627,061 (Barry, et al.), U.S. Pat. No. 5,633,435 (Barry, et al.), and U.S. Pat. No. 6,040,497 (Spencer, et al.) and aroA described in U.S. Pat. No. 5,094,945 (Comai) for glyphosate tolerance; a polynucleotide molecule encoding bromoxynil nitrilase (Bxn) described in U.S. Pat. No. 4,810,648 (Duerrschnabel, et al.) for Bromoxynil tolerance; a polynucleotide molecule encoding phytoene desaturase (crtI) described in Misawa et al. (1993) and Misawa et al. (1994) for norflurazon tolerance; a polynucleotide molecule encoding acetohydroxyacid synthase (AHAS, aka ALS) described in Sathasiivan et al. (1990) for tolerance to sulfonylurea herbicides; and both the PAT gene described in Wohlleben et al. (1988) and bar gene described in DeBlock et al. (1987) each of which provides glufosinate and bialaphos tolerance.

The regeneration, development, and cultivation of plants from various transformed explants are well documented in the art. This regeneration and growth process typically includes the steps of selecting transformed cells and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Cells that survive the exposure to the selective agent, or cells that have been scored positive in a screening assay, may be cultured in media that supports regeneration of plants. Developing plantlets are transferred to soil less plant growth mix, and hardened off, prior to transfer to a greenhouse or growth chamber for maturation.

This invention can be used with any transformable cell or tissue. By transformable as used herein is meant a cell or tissue that is capable of further propagation to give rise to a plant. Those of skill in the art recognize that a number of plant cells or tissues are transformable in which after insertion of exogenous DNA and appropriate culture conditions the plant cells or tissues can form into a differentiated plant. Tissue suitable for these purposes can include but is not limited to immature embryos, scutellar tissue, suspension cell cultures, immature inflorescence, shoot meristem, nodal explants, callus tissue, hypocotyl tissue, cotyledons, roots, and leaves. The Tomes et al. '783 patent, cited above, describes a method of treatment with a cytokinin followed by incubation for a period sufficient to permit undifferentiated cells in cotyledonary node tissue to differentiate into meristematic cells and to permit the cells to enter the phases between the G1 and division phases of development, which is stated to improve susceptibility for transformation.

Any suitable plant culture medium can be used. Suitable media include but are not limited to MS-based media (Murashige and Skoog, 1962) or N6-based media (Chu et al., 1975) supplemented with additional plant growth regulators including but not limited to auxins, cytokinins, ABA, and gibberellins. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Those of skill in the art are aware that media and media supplements such as nutrients and growth regulators for use in transformation and regeneration and other culture conditions such as light intensity during incubation, pH, and incubation temperatures that can be optimized for the particular variety of interest.

After an expression cassette is stably incorporated in transgenic plants and confirmed to be operable, it can be introduced into other plants of the same or another sexually compatible species by sexual crossing. Any of a number of standard breeding techniques can be used, depending upon the species to be crossed.

Seeds, Meal, Oil and Products Comprising Seeds, Meal and Oil

This invention also provides a container of over about 1000, more preferably about 20,000, and even more preferably about 40,000 seeds where over about 10%, more preferably about 25%, more preferably about 50%, and even more preferably about 75% or more preferably about 90% of the seeds are seeds derived from a plant of this invention.

This invention also provides a container of over about 10 kg, more preferably about 25 kg, and even more preferably about 50 kg seeds where over about 10%, more preferably about 25%, more preferably about 50%, and even more preferably about 75% or more preferably about 90% of the seeds are seeds derived from a plant of this invention.

Any of the plants or parts thereof of this invention may be harvested and, optionally, processed to produce a feed, meal, or oil preparation. A particularly preferred plant part for this purpose is harvested grain, but other plant parts can be harvested and used for stover or silage. In one embodiment the feed, meal, or oil preparation is formulated for ruminant animals. In such formulations, the increased oil content in grain and meal enabled by this invention provides “bypass fat” that is especially useful for providing increased caloric intake to dairy cows after calving with lower risk of acidosis. Methods to produce feed, meal, and oil preparations are known in the art. See, for example, U.S. Pat. Nos. 4,957,748; 5,100,679; 5,219,596; 5,936,069; 6,005,076; 6,146,669; and 6,156,227. The grain or meal of this invention may be blended with other grains or meals. In one embodiment, the meal produced from harvested grain of this invention or generated by a method of this invention constitutes greater than about 0.5%, about 1%, about 5%, about 10%, about 25%, about 50%, about 75%, or about 90% by volume or weight of the meal component of any product. In another embodiment, the meal preparation may be blended and can constitute greater than about 10%, about 25%, about 35%, about 50%, or about 75% of the blend by volume.

The corn oil and/or corn meal produced according to this invention may be combined with a variety of other ingredients. The specific ingredients included in a product will be determined according to the ultimate use of the product. Exemplary products include animal feed, raw material for chemical modification, biodegradable plastic, blended food product, edible oil, cooking oil, lubricant, biodiesel, snack food, cosmetics, and fermentation process raw material. Products incorporating the meal described herein also include complete or partially complete swine, poultry, and cattle feeds, pet foods, and human food products such as extruded snack foods, breads, as a food binding agent, aquaculture feeds, fermentable mixtures, food supplements, sport drinks, nutritional food bars, multi-vitamin supplements, diet drinks, and cereal foods.

The corn meal is optionally subjected to conventional methods of separating the starch and protein components. Such methods include, for example, dry milling, wet milling, high pressure pumping, or cryogenic processes. These and other suitable processes are disclosed in Watson (1987), the disclosure of which is hereby incorporated by reference.

Other monocot grains of this invention, including wheat, barley, sorghum and rice can similarly be processed or milled to produce feeds, flours, starches, meals, syrups, cereal products and fermented beverages well known to the art.

This invention is described further in the context of the following examples. These examples serve to illustrate further this invention and are not intended to limit the scope of the invention.

### EXAMPLES

Those of skill in the art will appreciate the many advantages of the methods and compositions provided by the present invention. The following examples are included to demonstrate embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention. All references cited herein are incorporated herein by reference to the extent that they supplement, explain, provide a background for, or teach methodology, techniques, or compositions employed herein.

### Example 1

### Cloning of the Lactobacillus delbreuckii Subspecies Bulgaricus pfk and pyk Genes

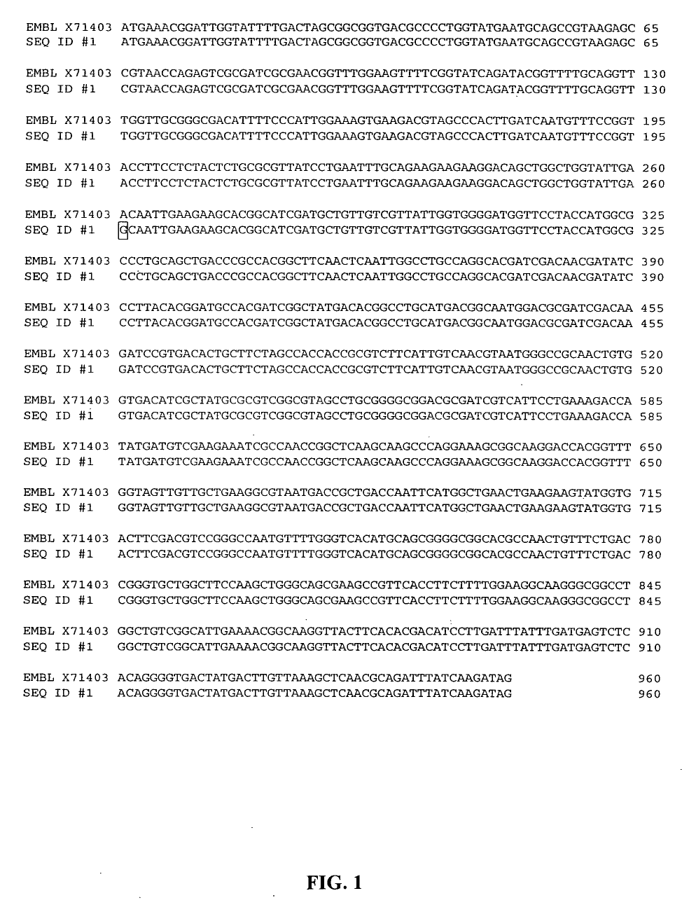

Lactobacillus delbreuckii subsp. bulgaricus (ATCC strain 11842) was obtained from ATCC (Manassas, Va.) and was grown in ATCC 416 broth. The L. delbreuckii subsp. bulgaricus pfk gene was PCR™ amplified as a 967 bp product from an aliquot of lysed culture using a 5′ primer (Oligo. # 17166) (SEQ ID NO:5) to introduce an AscI cloning site upstream of the pfk open reading frame (ORF) and a 3′ primer (Oligo. # 17167) (SEQ ID NO:6) to introduce an SbfI cloning site just downstream of the ORF. Similarly, the pyk gene was PCR™ amplified as a 1777 bp product from an aliquot of the lysed culture using a 5′ primer (Oligo. # 17168) (SEQ ID NO:7) to introduce an AscI cloning site just upstream of the pyk ORF and a 3′ primer (Oligo. # 17169) (SEQ ID NO:8) to introduce an SbfI cloning site downstream of the ORF. The pfk and pyk PCR products were each cloned into pCR2.1 by Topo TA cloning (Invitrogen, Carlsbad, Calif.). Clones were screened for the appropriate insert by PCR™ using the previously described oligos. Clones that were PCR-positive for the pfk or pyk genes were checked by restriction analysis to confirm the presence of the flanking cloning sites introduced by PCR™ and then by sequencing. FIG. 1 shows an alignment of the coding sequence of the pfk gene (SEQ ID NO:1) isolated from Lactobacillus delbreuckii subspecies bulgaricus ATCC strain 11842 with the published pfk gene sequence (EMBL accession # X71403). There was one difference between the sequence obtained above and the published sequence; the published sequence has an A at coding residue 261 while the gene isolated as described above has a G at that position. Alignment of the predicted PFK protein sequences (e.g SEQ ID NO:2) revealed that they were identical. The DNA sequence of the Lactobacillus delbreuckii subspecies bulgaricus pyk gene (SEQ ID NO:3) was also obtained and was identical to the published sequence (EMBL accession # X71403). Therefore the predicted protein sequence (SEQ ID NO:4) was identical to the published predicted PYK protein sequence.

TABLE 1

| | |
|---|---|
| Oligo. # 17166 | |
| 5′ AGGCGCGCCACCATGAAACGGATTGGT 3′ | (SEQ ID NO: 5) |
| Oligo. # 17167 | |
| 5′ CGCCTGCAGGCTATCTTGATAAATCTG 3′ | (SEQ ID NO: 6) |
| Oligo. # 17168 | |
| 5′ AGGCGCGCCACCATGAAAAAAACAAAG 3′ | (SEQ ID NO: 7) |
| Oligo. # 17169 | |
| 5′ CGCCTGCAGGTTACAGGTTTGAAAC 3′ | (SEQ ID NO: 8) |

### Example 2

### Construction of Embryo-Targeted Transformation Vectors

pMON72008

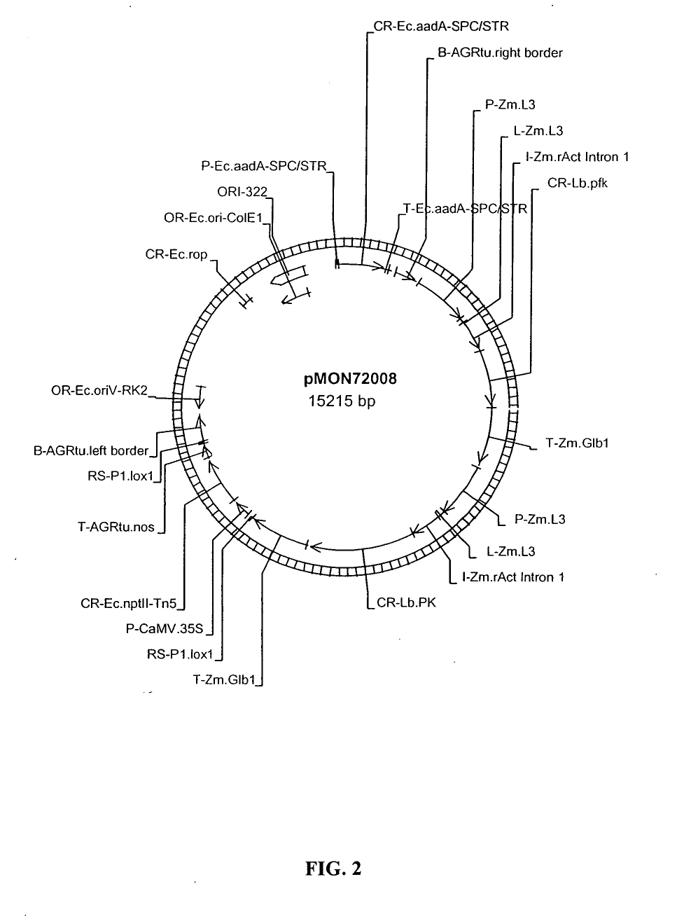

The 967 bp AscI/SbjI pjk gene described in Example 1 was cloned into the AscI/Sse8387I sites downstream of the maize L3 oleosin promoter (P-Zm.L3) and rice actin intron (I-Os.Act) sequences in the E. coli/Agrobacterium tumefaciens binary transformation vector pMON71055 to form pMON72004. Similarly, the 1777 bp AscI/SbfI pyk gene described in Example 1 was cloned into the AscI/Sse83871I sites downstream of the P-Zm.L3 and I-Os.Act sequences in the E. coli/A. tumefaciens binary transformation vector pMON71055 to form pMON72005. The pfk/pyk double gene construct (pMON72008) was prepared by isolating a 7165 bp PmeI/XbaI fragment from pMON72004 containing the pfk cassette, blunting the fragment using Pfu polymerase, and then cloning the blunt ended fragment into the PmeI site of pMON72005. The final construct, pMON72008 (FIG. 2) was confirmed by restriction analysis and DNA sequencing.

pMON79823

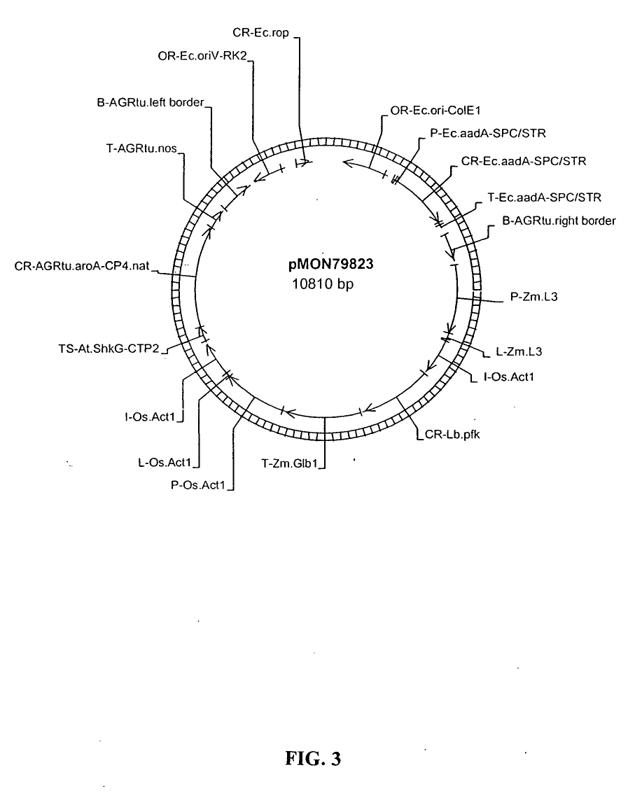

The 3616 bp PmeI/XbaI from pMON72004 was used to replace the 2145 bp PmeI/XbaI fragment from the germ expression vector pMON71273 to make pMON79823 (FIG. 3), containing the pjk gene driven by P-Zm.L3 with the I-Os.Act.

pMON79824

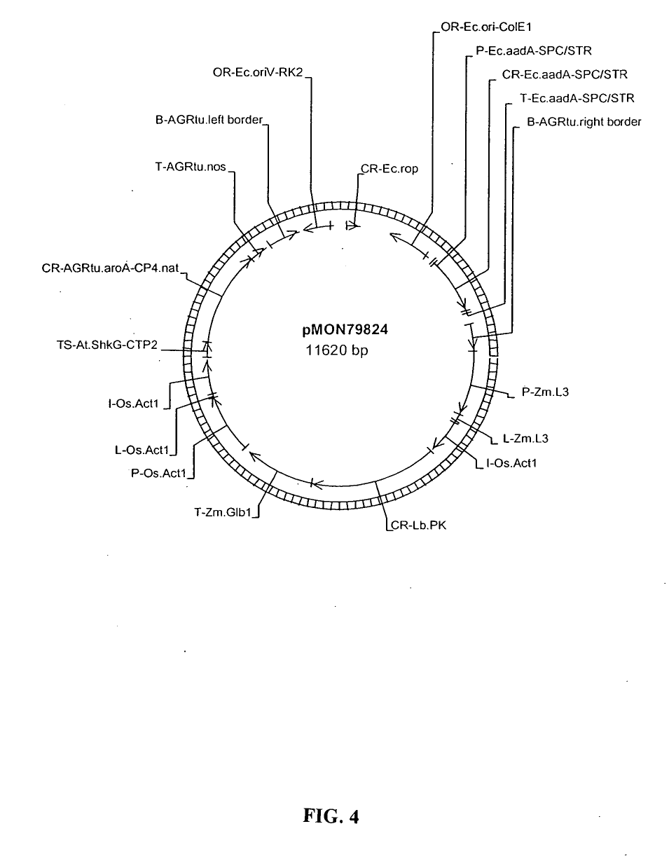

The 4426 bp PmeI/XbaI from pMON72005 was used to replace the 2145 bp PmeI/XbaI fragment from the germ expression vector pMON71273 to make pMON79824 (FIG. 4), containing the pyk gene driven by P-Zm.L3 with the I-Os.Act.

pMON79827

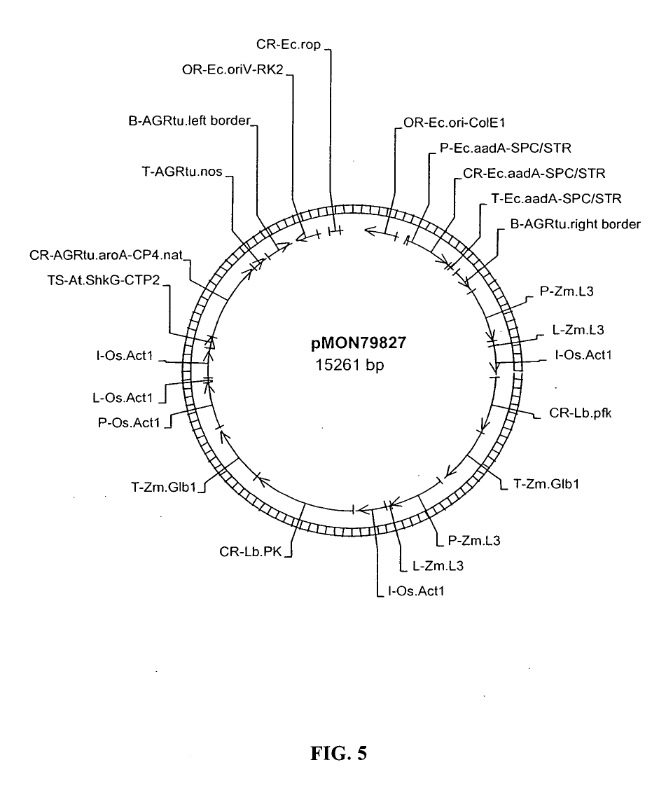

The 6809 PmeI/KspI fragment from pMON79824 was used to replace the 2358 bp SmaI/KspI fragment from pMON79823 to make pMON79827 (FIG. 5) containing the pjk and pyk genes, each driven by P-Zm.L3 with the I-Os.Act.

### Example 3

### Construction of Endosperm-Targeted Vectors

pMON72028

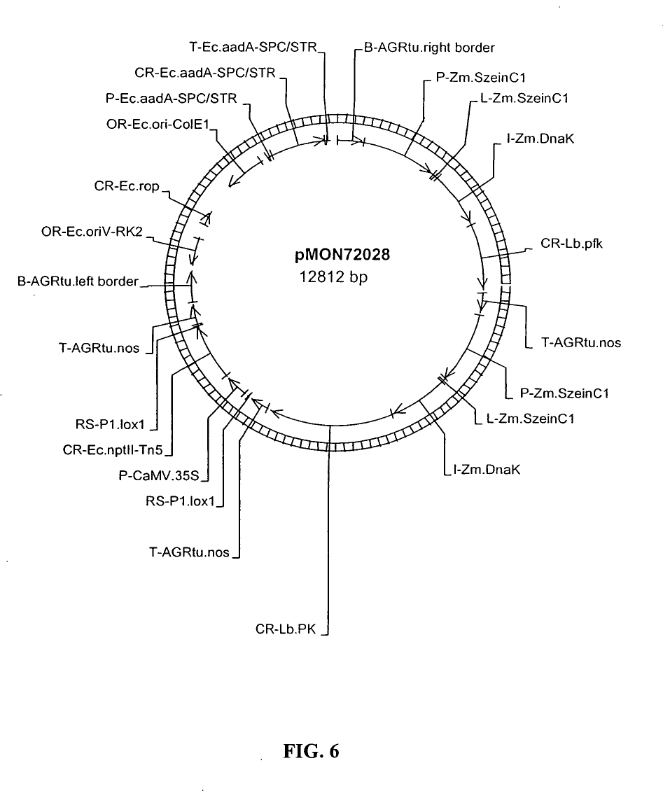

The 967 bp AscI/SbfI pfk gene described in Example 1 above was cloned into the AscI/Sse83871I sites downstream of the Zea mays Z27 promoter (P-Zm.Z27) and Z. mays Hsp70 intron (1-Zm.DnaK) sequences in pMON68203 to make pMON72012. Similarly, the 1777 bp AscI/SbfI pyk gene described in Example 1 above was cloned into the AscI/Sse83871I sites downstream of the P-Zm.Z27 and I-Zm.DnaK sequences in pMON68203 to make pMON72013. The vector for co-expression of the pfk and pyk genes was prepared by isolating the 3256 bp PmeI/EcoRI fragment containing the pjk expression cassette from pMON72012, blunt ending the fragment with Pfu polymerase, and cloning it into the PmeI site of pMON72013 (FIG. 5) to give pMON72015. To improve the stability of the pfk/pyk vector during A. tumefaciens transformation, the number of repetitive elements was reduced by replacing the 7318 bp PmeI/EcoRI vector backbone fragment of pMON72015 with the 5496 bp PmeI/EcoRI vector backbone fragment of pMON72021 to generate the final double gene transformation vector pMON72028 (FIG. 6).

pMON79832:

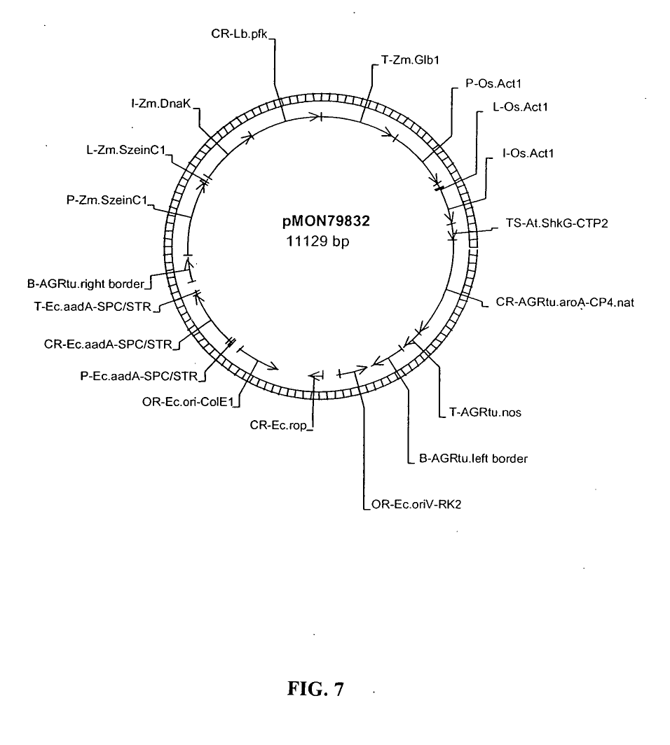

The 973 bp NotI/Sse8387I pjk gene described in Example 1 above was cloned into the Bsp120I/Sse83871I sites downstream of the P-Zm.Z27 and I-Zm.DnaK sequences in pMON71274 to make pMON79832 (FIG. 7), containing the pjk gene driven by P-Zm.Z27 with the I-Zm.DnaK.

pMON81470:

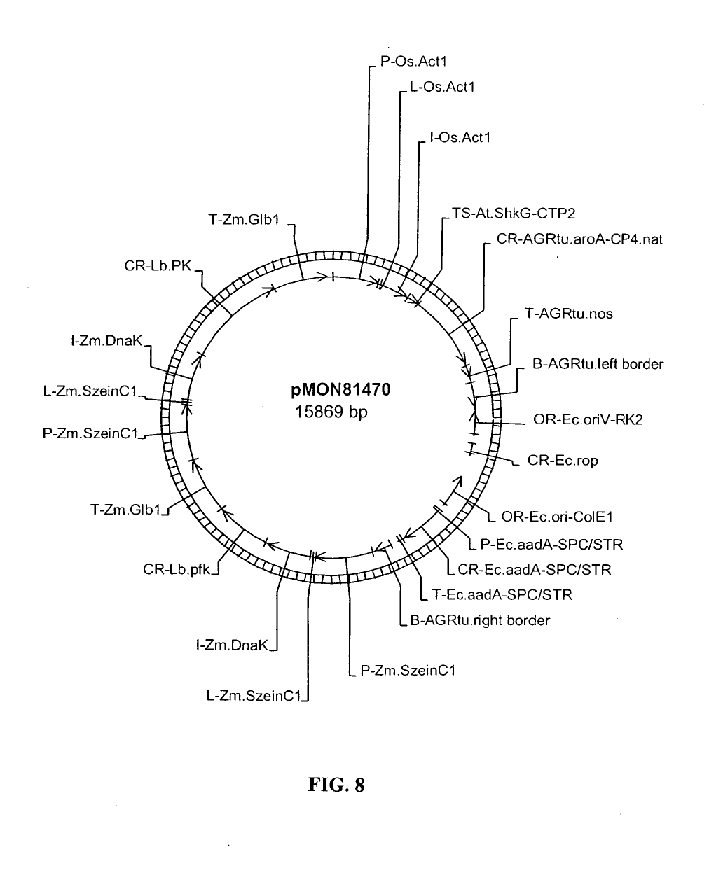

The 1783 bp NotI/Sse83871I pyk gene described in Example 1 above was cloned into the NotI/Sse83871I sites of pMON71274 downstream of the P-Zm.Z27 and I-Zm.DnaK sequences. The pyk gene cassette of the resulting vector was then cut out with AscI/SrfI and ligated into the MluI/SrfI sites of pMON79832 described above to make pMON81470 (FIG. 8), containing the pjk and pyk genes, each driven by P-Zm.Z27 with the I-Zm.DnaK.

pMON72029

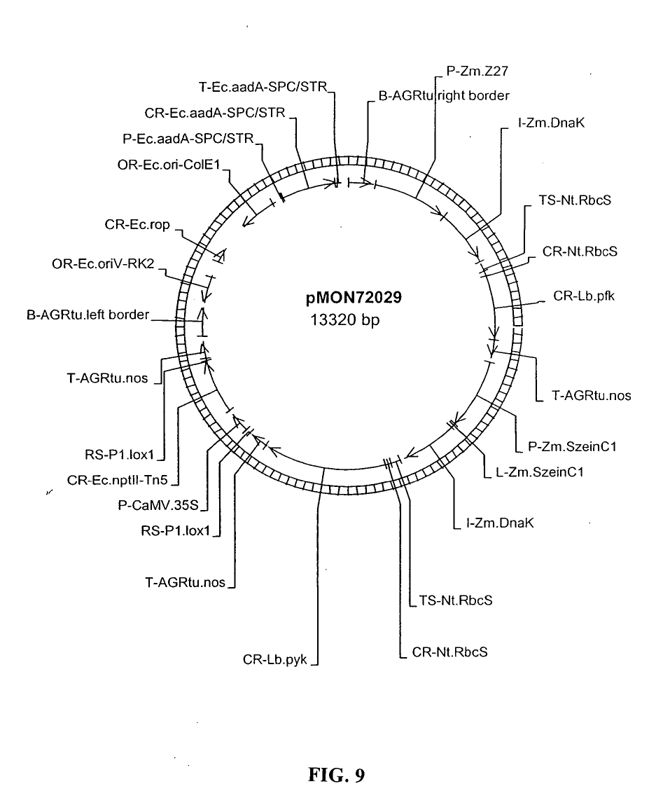

The 1199 bp AscI/Sse8387I DNA fragment containing the Nicotiana tabacum small subunit choroplast transit peptide (SSU-CTP) fused to the pjk gene from pMON72006 was cloned into the AscI/Sse8387I sites of pMON68203 to form pMON72017. Similarly, the 2041 bp AscI/Sse8387I fragment containing the N. tabacum SSU-CTP fused to the pyk gene from pMON72007 was cloned into the AscI/Sse8387I sites of pMON68203 to form pMON72019. The vector for co-expression of the pjk and pyk genes was prepared by isolating the 3204 bp PmeI/EcoRI DNA fragment containing the pjk expression cassette from pMON72017, blunt ending the fragment with Pfu polymerase, and cloning it into the PmeI site of pMON72019 to give pMON72020. To improve the stability of this pjk/pyk double gene vector during Agrobacterium tumefaciens transformation, the number of repetitive elements was reduced by replacing the 7135 bp PmeI/EcoRI vector backbone fragment of pMON72020 with the 5496 bp PmeI/EcoRI vector backbone fragment of pMON72021 to generate the final double gene transformation vector pMON72029 (FIG. 9).

pMON83715

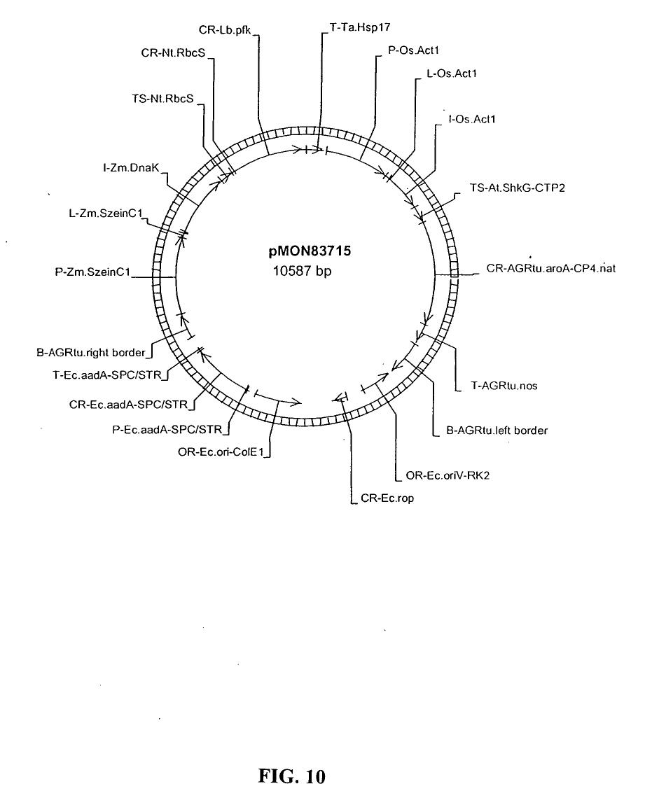

The 1.2 kb NotI/Sse83871I DNA fragment from pMON72017 containing the Nicotiana tabacum small subunit choroplast transit peptide (SSU-CTP) fused to the pfk gene was cloned into the NotI/Sse83871I sites of the glyphosate selection plasmid pMON93102 downstream of the Zea mays Z27 promoter (P-Zm.Z27) and Z. mays Hsp70 intron (1-Zm.DnaK) to make pMON83715 (FIG. 10).

### Example 4

### Transformation of Corn

Elite corn lines (Corn States Hybrid Serv., LLC, Des Moines, Iowa) are used for transformation in connection with this invention. These include LH59 (transformed with pMON72008, pMON72028, pMON72029), LH172 (transformed with pMON72008, pMON72028), and LH244 (transformed with pMON79823, pMON79824, pMON79827, pMON79832, pMON81470). Transformed explants are obtained through Agrobacterium tumefaciens-mediated transformation for all constructs except for pMON72029, which is obtained through microparticle bombardment. Plants are regenerated from transformed tissue. The greenhouse-grown plants are then analyzed for gene of interest expression levels as well as oil and protein levels.

### Example 5

### Analysis of Endosperm-Expressed Cytosol-Targeted PFK and PK Constructs

pMON72028

The construct pMON72028 was designed to produce cytosol-targeted expression of both the pfk and pyk genes in the endosperm. Mature kernels from the first generation were analyzed by PCR™ for the pjk and pyk transgenes. Sixty-seven events were analyzed by single kernel NMR and PCR™. 64 events were PCR-positive for the pyk transgene and 7 of these were also positive for the pjk transgene. Two events containing both genes demonstrated PCR-positive kernels that were statistically higher in whole kernel oil levels by comparison with the PCR-negative kernels (maximum increase of 0.73%, P=0.05).

The 7 events that were positive for both transgenes were planted in the field. NIT (near infrared transmittance) oil analysis revealed that for 3 events there was a significant difference in the mean whole kernel oil % for the pooled kernels from the segregating kanamycin-positive and -negative ears. These events, 62221, 71907 and 73131, had statistically significant increases in oil levels in the positive ears (1.2%, 0.8%, 0.5%, P=0.05) respectively. The oil levels were elevated in the remaining 4 events that were known to contain both transgenes, but the elevation was not significant at P=0.05.

Five events of construct pMON72028 containing both the pjk and pyk transgenes and their negative segregants were crossed to two different testers. The first tester was a conventional stiff stalk inbred and the second was a stiff stalk tester with a high oil phenotype (7.5% per se oil). The F1 hybrid seeds were planted at 6 locations in a design that resulted in separation of lines bearing the transgene from lines without a transgene by a range of male sterile hybrids. Entries were randomized differently at each location. Six ears were harvested by hand from the center of each plot, were shelled, and kernels were analyzed for oil, protein and starch by near infrared transmittance (NIT). Oil percent was increased in all 5 events from +0.5% to +1.1% with both testers (p<0.005).

pMON79832, F1

NMR oil analysis on F1 kernels from 26 events of pMON79832 in LH244 revealed that the pfk PCR-positive kernels from 9 of the 26 events tested were significantly (P=0.05) higher in whole kernel oil %, with a maximum increase of 0.95%. Considering all of the events together, students T-test revealed that the mean kernel oil % for the PCR-positive kernels (3.85%) was significantly higher (0.19%) (P<0.0001) than the mean for the PCR-negative kernels (3.66%). Analysis of the dissected endosperm tissue revealed that the PCR-positive kernels from 8 of the events had significantly (P=0.05) higher endosperm oil % than the negative kernels (maximum increase of 0.48%) and 7 events had significantly (P=0.05) higher total endosperm oil on a mg/kernel basis (maximum increase of 0.48 mg/kernel) despite the fact that the total endosperm dry wt was significantly (P=0.05) reduced (mean decrease of 8 mg/kernel, maximum decrease of 41 mg/kernel).

pMON81470, F1

NMR oil analysis on F1 kernels from 20 events of pMON79832 in LH244 revealed that the pjk PCR-positive kernels from 9 of the 20 events were significantly (P=0.05) higher in whole kernel oil %, with a maximum increase of 1.1%. Considering all of the events together, students T-test revealed that the mean kernel oil % for the PCR-positive kernels (4.47%) was significantly higher (0.4%, P<0.0001) than the mean for the negative kernels (4.07%). Analysis of the dissected endosperm tissue revealed that the PCR-positive kernels from 9 of the events had significantly (P=0.05) higher endosperm oil % than the negative kernels (mean increase of 0.3%, maximum increase of 0.62%) and 6 events had significantly higher total endosperm oil on a mg/kernel basis (mean increase, 0.28 mg/kernel; maximum increase, 0.48 mg/kernel) (P=0.05) despite the fact that the total endosperm dry wt was significantly reduced (mean decrease 30 mg/kernel) (P=0.05). Comparing these data with the data from the pjk alone construct (pMON79832) it appears that the magnitude of the oil difference is higher with the double gene construct pMON81470 and that there is a higher frequency of events with an increase in oil levels.

### Example 6

### Analysis of Endosperm Expressed Plastid-Targeted Construct

The construct pMON72029 was designed to produce plastid-targeted expression of both the pjk and pyk genes in corn endosperm. Reciprocal crosses were performed between the transgenic plants containing pMON72029 and non-transgenic LH59 and mature kernels were harvested from 62 separate events.

Single kernel analysis revealed that the mean endosperm oil concentration was significantly increased in 9 of the 13 events found to contain both transgenes by PCR™ (mean increase of 0.94%, maximum increase of 1.7%, P=0.05). None of the 3 events that contained only the pyk gene had elevated endosperm oil %. In terms of whole kernel oil %, 10 of the 13 events that contained both transgenes had significantly (P=0.05) increased whole kernel oil % (mean increase of 1.75%, maximum increase of 2.9%). In terms of the absolute quantity of oil/kernel, 4 of the 13 events with both genes had significantly (P=0.05) increased milligrams of oil/kernel (mean increase of 1.5 mg/kernel, maximum increase of 2.5 mg/kernel).

### Example 7

### Analysis of Germ-Expressed Cytosol-Targeted PFK and PK Constructs

pMON79823, F1

NMR analysis of the oil levels in the dissected pfk gene PCR-positive and -negative F1 kernels for 20 events from pMON79823 revealed that 7 of the 20 events analyzed had significantly (P=0.05) higher germ oil % in the positive kernels (mean increase of 1.7%, maximum increase of 5.8%). Also, 7 events had significantly (P=0.05) higher endosperm oil % in the positive kernels (mean increase of 0.14%, maximum increase of 0.34%), 4 of which were the same events that had the increase in germ oil %.

pMON79824, F1

NMR oil analysis of the pyk gene PCR-positive and -negative F1 kernels for 24 events from pMON79824 revealed that the germ oil % was unchanged in all but 1 of the events and, similarly, the whole kernel oil % was unchanged in all but 1 different event. A frequency of 1/24 for events with altered oil levels was no more than could be expected by random variation. Therefore, it appeared that the pyk transgene alone under these conditions did not affect oil levels.

pMON79827, F1

The pjk/pyk events were first screened for the pjk transgene. NMR oil analysis of the pfk gene PCR-positive and negative F1 kernels for 24 events from pMON79827 revealed that 10 out of the 20 events had significantly (P=0.05) increased germ oil % (mean increase of 2.23%, maximum increase of 5.39%). Also, despite the promoter being germ-enhanced, the endosperm oil % was increased in 5 events of the 20.

pMON72008

The construct pMON72008 was transformed in the elite variety LH172. Students T-test comparison of the mean germ oil % determined by NMR analysis of dissected mature germ tissue from 32 events revealed that the mean of all the pfk gene PCR-positive kernels across all the events was higher than the mean for the negative kernels by an absolute value of 2.59% and this difference was statistically significant (p=0.05). The maximum increase seen was 3.5%. The average of the total kernel oil % for the pfk gene PCR-positive kernels across all the events (2.89%) was slightly lower than the mean for the negative kernels (3.01%) although this difference was not significant at P=0.05.

Although the expression of the transgenes were directed by the L3 oleosin promoter, which is expressed in the germ tissue preferentially, there was a small but statistically significant increase in the average endosperm oil % across all the events for the pjk gene PCR-positive kernels as compared to the negative kernels (mean increase of 0.07%, maximum increase of 0.24%).

Further transgene expression analysis for pjk and pyk genes in the developing kernels from pMON72008 events was conducted by both western blotting analysis to test for protein expression and by enzyme assays. The western blotting analysis revealed that all 30 of the pjk gene PCR-positive events were found to express the PK protein, while 29 of the 30 were found to express the PFK protein. The PK protein was always expressed at a higher level than the PFK protein. The enzyme activity results agreed well with the western blot protein expression results. The elevation in PK activity was greater than the elevation in PFK activity, in agreement with the protein expression results.

### Example 8

### Construction of Transformation Vectors Expressing Propionibacterium freudenreichii Phosphofructokinase

Additional seed-specific constructs expressing the phosphofructokinase from Propionibacterium freudenreichii are generated. For endosperm cytosolic expression, the P. freudenreichii pjk gene (Genbank Accession #M67447) (SEQ ID NO:11) is amplified and is cloned downstream of the maize zein Z27 promoter optionally followed by the maize DnaK intron as an enhancer in a vector designed for maize transformation. For endosperm plastidial expression, the P. freudenreichii pfk gene (SEQ ID NO:11) is amplified and is cloned downstream of the maize zein Z27 promoter followed by the N. tabacum SSU CTP fused to the pfk gene in a vector designed for maize transformation. For germ cytosolic expression, the P. freudenreichii pfk gene (SEQ ID NO:11) is amplified and is cloned downstream of the barley PER1 promoter optionally followed by the maize DnaK intron as an enhancer in a vector designed for maize transformation. Transformed explants are obtained through transformation for all constructs. Plants are regenerated from transformed tissue. The greenhouse-grown plants are then analyzed for gene of interest expression levels as well as oil and protein levels.

All of the compositions and methods disclosed and claimed herein can be made and executed without undue experimentation in light of the present disclosure. While the compositions and methods of this invention have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### REFERENCES

The following references, to the extent that they provide exemplary procedural or other details supplementary to those set forth herein, are specifically incorporated herein by reference.
U.S. Pat. No. 4,518,584; U.S. Pat. No. 4,737,462; U.S. Pat. No. 4,810,648; U.S. Pat. No. 4,957,748; U.S. Pat. No. 5,094,945; U.S. Pat. No. 5,100,679; U.S. Pat. No. 5,196,525; U.S. Pat. No. 5,219,596; U.S. Pat. No. 5,290,924; U.S. Pat. No. 5,322,783; U.S. Pat. No. 5,359,142; U.S. Pat. No. 5,424,398; U.S. Pat. No. 5,424,412; U.S. Pat. No. 5,500,365; U.S. Pat. No. 5,538,880; U.S. Pat. No. 5,550,318; U.S. Pat. No. 5,563,055; U.S. Pat. No. 5,610,042; U.S. Pat. No. 5,627,061; U.S. Pat. No. 5,633,435; U.S. Pat. No. 5,641,876; U.S. Pat. No. 5,936,069; U.S. Pat. No. 6,005,076; U.S. Pat. No. 6,040,497; U.S. Pat. No. 6,146,669; U.S. Pat. No. 6,156,227; U.S. Pat. No. 6,265,638; U.S. Pat. No. 6,433,252; U.S. Pat. No. 7,012,171
Barany et al., Int. J. Peptide Protein Res., 30:705-739, 1987.
Bauer et al., Gene, 37:73, 1985.
Belanger and Kriz, Genet., 129:863-872, 1991.
Belanger and Kriz, Genetics, 129:863-872, 1991.
Bevan et al., Nucl. Acid Res., 11:369, 1983.
Bodanszky, In: Principles of Peptide Synthesis, Springer-Verlag, Heidelberg, 1984.
Burrell et al., Planta 194:95-101, 1994.
Callis et al., Genes Develop., 1:1183, 1987.
Chandler et al., The Plant Cell, 1:1175, 1989.
Chu et al., Scientia Sinica, 18:659, 1975.
Doehlert et al., Plant Physiol. 86:1013-1019, 1988.
DeBlock et al., EMBO J, 6:2513-2519, 1987.
della-Cioppa et al., Proc. Natl. Acad. Sci. USA, 83:6873-6877, 1986.
Ebert et al., Proc. Natl. Acad. Sci. USA, 84: 5745, 1987.
Gallie et al., The Plant Cell, 1:301, 1999.
Hudspeth et al., Plant Mol. Biol., 12:579, 1989.
Ingelbrecht et al., Plant Cell, 1:671-680, 1989.
Joshi, Nucl. Acid Res., 15:6643, 1987.
Klee et al., Mol. Gen. Genet., 210:437-442, 1987.
Kridl et al., Seed Sci. Res., 1:209-219, 1991.
Lawton et al., Plant Mol. Biol., 9:31F, 1987.
LeBras et al., Biochemie., 75:797-802, 1993.
LeBras et al., Eur. J. Biochem., 198:683-687, 1991.
Lopes et al., Mol. Gen. Genet., 247:603-613, 1995.
Lopes et al., Mol. Gen. Genet., 247:603-613, 1995.
Maniatis, et al., Molecular Cloning, A Laboratory Manual, Cold Spring Harbor Press, Cold Spring Harbor, N.Y., 1989.
Mazur, et al., Nucleic Acids Res., 13(7):2373-2386, 1985.
McElroy et al., Mol. Gen. Genet., 231(1):150-160, 1991.
Merrifield, J. Am. Chem. Soc., 85:2149-2154, 1963.
Misawa et al, Plant J, 4:833-840, 1993.
Misawa et al, Plant J, 6:481-489, 1994.
Murashige and Skoog, Physiol. Plant, 15:473-497, 1962.
Odell et al., Nature, 313:810, 1985.
PCT Appln. WO 95/06128
PCT Appln. WO 99/67392
Pedersen et al., Cell, 29:1015-1026, 1982.
Plaxton, Ann. Rev. Plant Physiol. Plant Mol. Biol., 47:185-214, 1996.
Recombinant DNA Part D, Methods in Enzymology, Vol. 153, Wu and Grossman, eds., Academic Press, 1987.
Richins et al., Nucleic Acids Res., 20:8451, 1987.
Russell et al., Transgenic Res., 6(2):157-168, 1997.
Sambrook et al., In: Molecular cloning: a laboratory manual, 2^{nd} Ed., Cold Spring Harbor Laboratory Press, Cold Spring Harbor, N.Y., 1989.
Sathasiivan et al., Nucl. Acids Res., 18:2188-2193, 1990.
Stacey et al., Plant Mol. Biol., 31:1205-1216, 1996.
Sullivan et al., Mol. Gen. Genet., 215:431, 1989.
Thomas et al., Biochem. J. 322:111-117, 1997.
Turner and Foster, Molecular Biotech., 3:225, 1995.
Vasil et al., Plant Physiol., 91:5175, 1989.
Walder et al., Gene, 42:133, 1986.
Walker et al., Proc. Natl. Acad. Sci. USA, 84:6624, 1987.
Wang et al., Mol. Cell. Biol., 12:3399, 1992.
Watson and Ramstad, In: Corn: Chemistry and Technology, Ch. 11-12, Amer. Assoc. Cereal Chemist, Inc., St. Paul, Minn., 1987.
Wohlleben et al., Gene, 70:25-37, 1988.
Wood et al., J. Exp. Bot. 50:16, 1999.
Wood et al., Can. J. Bot. 80:993-1001, 2002.
Yang et al., Proc. Natl. Acad. Sci. USA, 87:4144, 1990.

What is claimed is:

1. A method of producing a monocot plant having increased oil in its seed, comprising introducing into said plant a polynucleotide encoding a phosphofructokinase, operably linked to a seed-enhanced promoter whereby the oil content of the seed is increased as compared to a seed of an isogenic plant lacking the nucleic acid sequence.

2. The method of claim 1, wherein the polynucleotide encoding a phosphofructokinase comprises a sequence other than SEQ ID NO:9 or SEQ ID NO:13.

3. The method of claim 1, wherein the polynucleotide encoding a phosphofructokinase is operably linked to a polynucleotide encoding a plastid transit peptide except when said seed-enhanced promoter is an embryo-enhanced promoter.

4. The method of claim 1, wherein the polynucleotide comprises a nucleic acid sequence selected from the group consisting of:
(a) a nucleic acid sequence of SEQ ID NO:1 or SEQ ID NO:11; and
(b) a nucleic acid sequence that encodes the polypeptide sequence of SEQ ID NO:2, or SEQ ID NO:12.

5. The method of claim 4, wherein the polynucleotide comprises a nucleic acid sequence that hybridizes to the sequence of (a) or (b) or a complement thereof under high stringency conditions of about 0.2×SSC and 65° C.

6. The method of claim 4, wherein the plant further comprises a polynucleotide encoding a pyruvate kinase operably linked to a seed-enhanced promoter.

7. The method of claim 6, wherein the polynucleotide encoding a pyruvate kinase comprises a nucleic acid sequence selected from the group consisting of:
(a) a nucleic acid sequence comprising the sequence of SEQ ID NO:3; and
(b) a nucleic acid sequence that encodes the polypeptide sequence of SEQ ID NO:4.

8. The method of claim 7, wherein the polynucleotide comprises a nucleic acid sequence that hybridizes to the sequence of (a) or (b) or a complement thereof under high stringency conditions of about 0.2×SSC and 65° C.

9. The method of claim 1, wherein the plant is a monocot selected from the group consisting of corn (Zea mays), rice (Oryza sativa), barley (Hordeum vulgare), millet (Panicum miliaceum), rye (Secale cereale), wheat (Triticum aestivum), and sorghum (Sorghum bicolor).

10. The method of claim 1, wherein the promoter is selected from the group consisting of embryo-enhanced promoters, endosperm-enhanced promoters and embryo- and endosperm-enhanced promoters.

11. A monocot plant comprising a polynucleotide encoding a phosphofructokinase, operably linked to a seed-enhanced promoter.

12. The plant of claim 11, wherein the polynucleotide encoding a phosphofructokinase comprises a sequence other than SEQ ID NO:9 or SEQ ID NO:13.

13. The plant of claim 11, wherein the polynucleotide encoding a phosphofructokinase is linked to a polynucleotide encoding a plastid transit peptide except when said seed-enhanced promoter is an embryo-enhanced promoter.

14. A monocot plant cell comprising a polynucleotide encoding a phosphofructokinase, operably linked to a seed-enhanced promoter.

15. A seed produced from the plant of claim 11, comprising a polynucleotide encoding a phosphofructokinase according to claim 7.

16. A meal produced from the seed of claim 15 comprising a polynucleotide encoding a phosphofructokinase according to claim 11.

17. An animal feed composition produced from the seed of claim 15 comprising a polynucleotide encoding a phosphofructokinase according to claim 11.

18. A human food composition produced from the seed of claim 15 comprising a polynucleotide encoding a phosphofructokinase according to claim 11.

19. An animal feed composition comprising the meal of claim 16 comprising a polynucleotide encoding a phosphofructokinase according to claim 11.

20. A method of making a monocot plant oil comprising the steps of:
a) growing a transformed monocot plant comprising a polynucleotide encoding a phosphofructokinase operably linked to a seed-enhanced promoter, to produce seed; and
b) processing the seed to obtain the oil.

21. The method of claim 20, wherein the polynucleotide encoding a phosphofructokinase comprises a sequence other than SEQ ID NO:9 or SEQ ID NO:13.

22. The method of claim 20, wherein the polynucleotide encoding a phosphofructokinase is linked to a polynucleotide encoding a plastid transit peptide except when said seed-enhanced promoter is an embryo-enhanced promoter.
